(19) United States
(12) Patent Application Publication
(10) Pub. No.: US 2014/0228230 A1
(43) Pub. Date: Aug. 14, 2014

(54) RESISTANCE GENE TO XANTHOMONAS AXONOPODIS IN SOYBEANS

(71) Applicants: Sung Yun Kim, Busan (KR); Yong Chul Kim, Gyeongsangnam-do (KR); Hyun Kyung Kim, Busan (KR); Jung Min Kim, Daejeon (KR); In Soo Choi, Busan (KR); Young Whan Choi, Busan (KR); Jum Soon Kang, Gyeongsangnam-do (KR); Sun Tae Kim, Busan (KR); Young Hoon Park, Gyeongsangnam-do (KR); Keun Ki Kim, Gyeongsangnam-do (KR); Ki Won Oh, Daegu (KR); Young Hoon Lee, Gyeongsangnam-do (KR); Chan Sik Jung, Busan (KR); Jong Mim Ko, Daegu (KR); In Youl Baek, Gyeongsangnam-do (KR); Keum Yong Park, Gyeonggi-do (KR); Hee Yeong Chung, Daegu (KR)

(72) Inventors: Sung Yun Kim, Busan (KR); Yong Chul Kim, Gyeongsangnam-do (KR); Hyun Kyung Kim, Busan (KR); Jung Min Kim, Daejeon (KR); In Soo Choi, Busan (KR); Young Whan Choi, Busan (KR); Jum Soon Kang, Gyeongsangnam-do (KR); Sun Tae Kim, Busan (KR); Young Hoon Park, Gyeongsangnam-do (KR); Keun Ki Kim, Gyeongsangnam-do (KR); Ki Won Oh, Daegu (KR); Young Hoon Lee, Gyeongsangnam-do (KR); Chan Sik Jung, Busan (KR); Jong Mim Ko, Daegu (KR); In Youl Baek, Gyeongsangnam-do (KR); Keum Yong Park, Gyeonggi-do (KR); Hee Yeong Chung, Daegu (KR)

(73) Assignee: PUSAN NATIONAL UNIVERSITY INDUSTRY- UNIVERSITY COOPERATION FOUNDATION, Pusan (KR)

(21) Appl. No.: 14/118,494

(22) PCT Filed: Oct. 31, 2011
(86) PCT No.: PCT/KR2011/008227

§ 371 Date: Mar. 6, 2014

(30) Foreign Application Priority Data
Oct. 31, 2010 (KR) 10-2010-0107385

(51) Int. Cl. C12Q 1/68

(52) U.S. Cl. CPC C12Q 1/6895
USPC 506/9; 435/6.11; 435/6.12; 536/23.6; 536/23.2; 536/24.33; 506/16

(57) ABSTRACT

The present invention relates to a marker composition for diagnosing resistance to bacterial blight of soybean; a composition for diagnosing resistance to bacterial blight of soybean, comprising a primer which specifically binds to a marker gene; a diagnostic kit for diagnosing resistance to bacterial blight of soybean, comprising the composition; and a method for diagnosing resistance to bacterial blight of soybean. As described above, with the use of the marker gene for diagnosing resistance to bacterial blight of soybean according to the present invention, it is possible to breed varieties that are resistant to bacterial blight of soybean, thus providing disease-resistant and high-quality, superior varieties.

### TECHNICAL FIELD

The present invention relates to a marker composition for diagnosing resistance to bacterial blight of soybean; a composition for diagnosing resistance to bacterial blight of soybean, comprising a primer which specifically binds to a marker gene; a diagnostic kit for diagnosing resistance to bacterial blight of soybean, comprising the composition; and a method for diagnosing resistance to bacterial blight of soybean.

### BACKGROUND ART

Soybean is an important food crop in the world and is also an important crop in the cropping system for the maintenance and improvement of soil fertility.

It was reported that bacterial blight of soybean is caused by Xanthomonas axonopodis pv. glycines and reduces the number of seeds per pod and the one hundred seed weight, resulting in a decrease in yield (Arun et al. 1993, Weber et al. 1966, Lee. 1999) or reduces the content of protein, one of the major nutrients of the seed (Hartwig & Johnson. 1953). The pathogen causing the bacterial blight of soybean overwinters in seeds or tissues of diseased plants, occurs in primary leaves of young seedlings, and spreads upward along the leaves. In particular, the pathogen of bacterial blight of soybean occurs at the maximum in a hot and humid environment and has thermophilic properties that the progress of the disease is not interrupted by hot weather. The bacterial blight of soybean is distributed throughout the world due to its high pathogenicity. In Korea, since the first report in 1970s, the bacterial blight of soybean has recently spread across the country. The occurrence of bacterial blight of soybean was observed in 90% of a total of 106 field tests all over the country carried out for two years in 1997 and 1998 (Lee. 1999).

However, any medicines for preventing bacterial blight of soybean or resistant varieties have not yet been developed, and excessive use of chemicals for preventing the occurrence of bacterial blight of soybean, such as agricultural chemicals, causes environmental pollution and has an adverse effect on the human body.

Therefore, the present inventors have made efforts to develop resistant varieties for bacterial blight of soybeans and found marker genes for diagnosing resistance to bacterial blight of soybean, thus completing the present invention.

### DISCLOSURE

### Technical Problem

An object of the present invention is to provide a marker composition for diagnosing resistance to bacterial blight of soybean.

Another object of the present invention is to provide a composition for diagnosing resistance to bacterial blight of soybean comprising a substance that measures the level of expression of the marker.

Still another object of the present invention is to provide a diagnostic kit for diagnosing resistance to bacterial blight of soybean, comprising the composition, and a method for diagnosing resistance to bacterial blight of soybean.

### Technical Solution

To achieve the above objects, the present invention provides a marker composition for diagnosing resistance to bacterial blight of soybean, comprising at least one gene selected from the group consisting of AB052784 (SEQ ID NO: 1), AF022780 (SEQ ID NO: 2), BI944059 (SEQ ID NO: 3), U13987 (SEQ ID NO: 4), D86929 (SEQ ID NO: 5), BE823110 (SEQ ID NO: 6), FG999662 (SEQ ID NO: 7), CX710871 (SEQ ID NO: 8 or 18), AF055369 (SEQ ID NO: 9), CS226295 (SEQ ID NO: 10), BU550410 (SEQ ID NO: 11), EV281281 (SEQ ID NO: 12), EV276436 (SEQ ID NO: 13), EF551167 (SEQ ID NO: 14), EH258681 (SEQ ID NO: 15), CX709057 (SEQ ID NO: 16), and CX702069 (SEQ ID NO: 17).

Moreover, the present invention provides a composition for diagnosing resistance to bacterial blight of soybean, comprising at least one primer selected from the group consisting of SEQ ID NOs: 19 to 52.

Furthermore, the present invention provides a diagnostic kit for diagnosing resistance to bacterial blight of soybean, comprising the composition; and a method for diagnosing resistance to bacterial blight of soybean.

### Advantageous Effects

With the use of marker genes for diagnosing resistance to bacterial blight of soybean according to the present invention, it is possible to breed varieties that are resistant to bacterial blight of soybean, thus providing disease-resistant and high-quality, superior varieties.

### DESCRIPTION OF DRAWINGS

FIG. 1 shows the microarray data and Real-time PCR results of a gene AB052784 that is resistant to bacterial blight of soybean according to the present invention.

FIG. 2 shows the microarray data and Real-time PCR results of a gene AF022780 that is resistant to bacterial blight of soybean according to the present invention.

FIG. 3 shows the microarray data and Real-time PCR results of a gene BI944059 that is resistant to bacterial blight of soybean according to the present invention.

FIG. 4 shows the microarray data and Real-time PCR results of a gene U13987 that is resistant to bacterial blight of soybean according to the present invention.

FIG. 5 shows the microarray data and Real-time PCR results of a gene D86929 that is resistant to bacterial blight of soybean according to the present invention.

FIG. 6 shows the microarray data and Real-time PCR results of a gene BE823110 that is resistant to bacterial blight of soybean according to the present invention.

FIG. 7 shows the microarray data and Real-time PCR results of a gene FG999662 that is resistant to bacterial blight of soybean according to the present invention.

FIG. 8 shows the microarray data and Real-time PCR results of a gene CX710871 that is resistant to bacterial blight of soybean according to the present invention.

FIG. 9 shows the microarray data and Real-time PCR results of a gene AF055369 that is resistant to bacterial blight of soybean according to the present invention.

FIG. 10 shows the microarray data and Real-time PCR results of a gene CS226295 that is resistant to bacterial blight of soybean according to the present invention.

FIG. 11 shows the microarray data and Real-time PCR results of a gene BU550410 that is resistant to bacterial blight of soybean according to the present invention.

FIG. 12 shows the microarray data and Real-time PCR results of a gene EV281281 that is resistant to bacterial blight of soybean according to the present invention.

FIG. 13 shows the microarray data and Real-time PCR results of a gene EV276436 that is resistant to bacterial blight of soybean according to the present invention.

FIG. 14 shows the microarray data and Real-time PCR results of a gene EF551167 that is resistant to bacterial blight of soybean according to the present invention.

FIG. 15 shows the microarray data and Real-time PCR results of a gene EH258681 that is resistant to bacterial blight of soybean according to the present invention.

FIG. 16 shows the microarray data and Real-time PCR results of a gene CX709057 that is resistant to bacterial blight of soybean according to the present invention.

FIG. 17 shows the microarray data and Real-time PCR results of a gene CX702069 that is resistant to bacterial blight of soybean according to the present invention.

FIG. 18 show the analysis results of the degree of resistance to bacterial blight of soybean depending on the concentration of sucrose treated.

### MODE FOR INVENTION

Hereinafter, the present invention will be described in detail.

In an aspect, the present invention provides a marker composition for diagnosing resistance to bacterial blight of soybean, comprising at least one gene selected from the group consisting of AB052784 (Glycine max mRNA for nitrate transporter NRT1-1, SEQ ID NO: 1), AF022780 (Glycine max nitrate reductase (BCNR-A), SEQ ID NO: 2), BI944059 (sq64f11.y1 Gm-c1048 Glycine max cDNA clone GENOME SYSTEMS CLONE ID: Gm-c1048-262 5- similar to TR:O82161 O82161 PHI-1 PROTEIN, SEQ ID NO: 3), U13987 (Glycine max inducible nitrate reductase 2 (INR2), SEQ ID NO: 4), D86929 (Glycine max mRNA for uricase, SEQ ID NO: 5), BE823110 (GM700020A10D10 Gm-r1070 Glycine max cDNA clone Gm-r1070-7843 3-, SEQ ID NO: 6), FG999662 (GLPAC22TF JCVI-SOY1 Glycine max cDNA 5-, SEQ ID NO: 7), CX710871 (gmrtDrNS01_{—}24-D_M13R_D08 058.s3 Water stressed 48 h segment 1 gmrtDrNS01 Glycine max cDNA 3-, SEQ ID NO: 8 (CX710871-Glyma15g08800.1) or SEQ ID NO: 18 (CX710871-Glyma15g08800.2)), AF055369 (Glycine max nitrate reductase (nr2) gene, SEQ ID NO: 9), CS226295 (Sequence 78 from Patent WO2005098015, SEQ ID NO: 10), BU550410 (GM880017B20A09 Gm-r1088 Glycine max cDNA clone Gm-r1088-6186 3-, SEQ ID NO: 11), EV281281 (GLNB304TF JCVI-SOY3 Glycine max cDNA 5-, SEQ ID NO: 12), EV276436 (GLMCT07TF JCVI-SOY2 Glycine max cDNA 5-, SEQ ID NO: 13), EF551167 (Glycine max dehydration-responsive element binding protein 7, SEQ ID NO: 14), EH258681 (JGI_ACBU2446.fwd ACBU Phakopsora pachyrhizi infected soybean leaf tissue 6-8 days post inoculation with TW72-1 urediniospores Glycine max cDNA clone ACBU2446 5-, SEQ ID NO: 15), CX709057 (gmrtDrNS01_{—}03-D_M13R_B04_{—}030.s4 Water stressed 48 h segment 2 gmrtDrNS01 Glycine max cDNA 3-, SEQ ID NO: 16), and CX702069 (gmrtDrNS01_{—}01-B_T3_D_{—}02 010.s0 Water stressed gmrtDrNS01 Glycine max cDNA 3-, SEQ ID NO: 17).

The gene is derived from soybean and includes both genomic DNA and cDNA. Moreover, variants of the above sequences are within the scope of the present invention. Specifically, the gene may comprise a nucleotide sequence having a sequence homology of more than 70% with the nucleotide sequences of SEQ ID NOs: 1 to 17, preferably more than 80%, more preferably more than 90%, most preferably more than 95%.

In another aspect, the present invention provides a composition for diagnosing resistance to bacterial blight of soybean, comprising a substance that measures the level of expression of the marker.

Preferably, the composition for diagnosing resistance to bacterial blight of soybean according to the present invention provides at least one primer selected from the group consisting of SEQ ID NOs: 19 to 52.

As used herein, the term “primer” refers to a short nucleic acid sequence having a free 3′ hydroxyl group, which forms a base pair with a complementary template so as to serve as a starting point for the replication of the template strand. The primer initiates DNA synthesis in the presence of four different deoxy-nucleotide triphosphates (dNTP) and an agent for polymerization (DNA polymerase or reverse transcriptase) in an appropriate buffer and at a suitable temperature.

The primer according to the present invention refers to a primer that can hybridize to any complementary marker gene for diagnosing resistance to bacterial blight of soybean and may preferably be a primer or a pair of primers (sense (forward) and antisense(reverse) nucleic acids) having 7 to 50 nucleotide sequences capable of amplifying at least one gene selected from the group consisting of AB052784 (SEQ ID NO: 1), AF022780 (SEQ ID NO: 2), BI944059 (SEQ ID NO: 3), U13987 (SEQ ID NO: 4), D86929 (SEQ ID NO: 5), BE823110 (SEQ ID NO: 6), FG999662 (SEQ ID NO: 7), CX710871 (SEQ ID NO: 8 or SEQ ID NO: 18), AF055369 (SEQ ID NO: 9), CS226295 (SEQ ID NO: 10), BU550410 (SEQ ID NO: 11), EV281281 (SEQ ID NO: 12), EV276436 (SEQ ID NO: 13), EF551167 (SEQ ID NO: 14), EH258681 (SEQ ID NO: 15), CX709057 (SEQ ID NO: 16), and CX702069 (SEQ ID NO: 17), and sequences disclosed in table 1. The primer may include additional features that do not change the basic properties of a primer that acts as a starting point of for DNA synthesis. Moreover, the nucleic acid sequence of the primer according to the present invention may include a label detectable, either directly or indirectly by spectroscopic, photochemical, biochemical, immunochemical, or chemical means, if necessary. Examples of the label include enzymes (e.g., horseradish peroxidase, alkaline phosphatase), radioisotope (e.g., 32P), fluorescent dyes, chemical groups (e.g., biotin), etc. The pair of primers of the invention includes all combinations of primer pairs consisting of forward and reverse primers that recognize the sequence of the target gene, preferably a pair of primers providing analysis results with specificity and sensitivity.

Moreover, the composition for diagnosing resistance to bacterial blight of soybean according to the present invention may include a probe that specifically binds to the gene.

As used herein, the term “probe” refers to a nucleic acid fragment such as RNA or DNA capable of specifically binding to mRNA, ranging from several to hundreds of bases in length, and the probe is labeled so as to detect the presence or absence of a specific mRNA. The probe may be prepared in the form of oligonucleotide probe, single stranded DNA probe, double stranded DNA probe, RNA probe, etc.

The primer or probe of the present invention may be chemically synthesized by a phosphoramidite solid support method or other methods well known in the art. These nucleic acid sequences may also be modified using any means known in the art. Non-limiting examples of such modifications include methylation, capsulation, replacement of one or more native nucleotides with analogues thereof, and inter-nucleotide modifications, for example, modifications to uncharged conjugates (e.g., methyl phosphonate, phosphotriester, phosphoramidate, carbamate, etc.) or charged conjugates (e.g., phosphorothioate, phosphorodithioate, etc.).

Moreover, the composition for diagnosing resistance to bacterial blight of soybean according to the present invention may include a substance capable of measuring the level of a protein that is encoded by the gene. The substance includes an “antibody” such as a polyclonal antibody, a monoclonal antibody, and a recombinant antibody, etc. The antibodies can be easily prepared by those skilled in the art using a known method. The polyclonal antibody can be prepared by any methods well known in the art, in which a protein antigen encoded by the gene is injected to an animal, and then the blood is collected from the animal to obtain the serum containing the antibody. The polyclonal antibody may be produced from any animal species host hosts including goats, rabbits, sheep, monkey, horse, swine, cattle, dog, etc. The monoclonal antibody may be produced by any methods well known in the art such as a hybridoma method (see. Kohler and Milstein (1976), European Journal of Immunology 6: 511-519) and a phage antibody library (Clackson et al, Nature, 352: 624-628, 1991; Marks et al, J. Mol. Biol., 222:58, 1-597, 1991). Moreover, the antibody of the present invention includes functional fragments of antibody molecules, as well as a complete form having two full-length light chains and two full-length heavy chains. The functional fragment of antibody molecules means a fragment having at least an antigen-binding function and includes Fab, F(ab′), F(ab′)2, Fv, etc.

In still another aspect, the present invention provides a diagnostic kit for diagnosing resistance to bacterial blight of soybean, comprising the composition for diagnosing resistance to bacterial blight of soybean.

The diagnostic kit of the present invention may further comprise one or more compositions, solutions or instruments, which are suitable for analysis methods. Preferably, the diagnostic kit may be a kit for detecting a diagnostic marker that includes essential elements required to perform RT-PCR. An RT-PCR kit may include test tubes or other suitable containers, reaction buffers (varying in pH and magnesium concentrations), deoxynucleotides (dNTPs), enzymes such as Taq-polymerase and reverse transcriptase, DNase and RNase inhibitors, DEPC water, and sterile water, in addition to a pair of primers specific for the marker gene. Moreover, the RT-PCR kit may include a pair of primers specific for a gene used as a quantitative control. Furthermore, the RT-PCR kit may preferably be a DNA chip kit that includes all essential elements required to perform microarray analysis. The DNA chip kit may include a substrate to which a gene or cDNA or oligonucleotide corresponding to a fragment thereof is attached.

Furthermore, when the substance for measuring the level of protein is preferably an antibody in the present invention, the diagnostic kit may include essential elements required to perform ELISA. The ELISA kit may include a reagent for detecting a bound antibody, such as a labeled secondary antibody, chromophores, an enzyme (e.g., conjugated with an antibody), and a substrate thereof. Moreover, the kit may further include an antibody specific for a protein as a quantitative control. Furthermore, the protein measurement using an antibody in the present invention may include a diagnostic method using a protein chip. The protein chip may include a substrate with an attached antibody. In addition, the protein measurement using an antibody may include a substrate for detecting surface plasmon resonance (SPR).

As used herein, the term “microarray” refers to a high-density array of groups of polynucleotides or polypeptides immobilized on a substrate. Here, each group of the polynucleotides or polypeptides is a microarray immobilized in a predetermined region of the substrate. The microarray may be a DNA chip, a peptide nucleic acid (PNA) chip, or a protein chip, but not limited thereto. The microarray is well known in the art. Examples of the microarrays are disclosed in U.S. Pat. Nos. 5,445,934 and 5,744,305, the disclosures of which are incorporated herein by reference.

In yet another aspect, the present invention provides a method for diagnosing resistance to bacterial blight of soybean.

Hereinafter, the present invention will be described in detail with reference to Examples. However, the following Examples are only intended to illustrate the present invention, and the scope of the present invention is not limited to the following Examples.

### Example 1

### Inoculation with Pathogen of Bacterial Blight of Soybean

An F7 system of Saturn//Lee/Seonheuk was selected for microarray analysis by inoculating major soybean breeding lines and RIL 160 system with the pathogen of bacterial blight (8ra) and examining the infection of the disease.

The pathogen of bacterial blight used in the present invention was obtained in a manner that pathogenic bacteria kept at −70° C. were cultured in a potato dextrose agar(PDA) medium for two days, subcultured, and then cultured in bulk for inoculation.

The PDA medium was prepared in the following manner. First, 35 g of PDA powder was added to 1 L of distilled water and autoclaved at 121° C. for 15 minutes. When the autoclaved medium was cooled to about 50 to 55° C., about 15 to 20 mL of the medium was distributed into each Petri dish, solidified, and kept under refrigeration at 4° C. The PDA medium under refrigeration was placed in an incubator at 28° C. for 2 hours before use to reduce the temperature shock. Then, the bacteria were streaked (Bloom et al., 1996) and cultured at 28° C. for 1.5 to 2 days. The colonies of the bacterial blight pathogen cultured at 28° C. for about 2 days were pale yellow on the medium. About 2 mL of distilled water was added to each Petri dish, and the resulting bacteria were collected using a triangular scraper, suspended, diluted with distilled water to an absorbent of 0.3 to 0.5 (108 colony forming units (cfu) per milliliter) at a wavelength of 600 nm using a UV-visible spectrophotometer, and then used for the inoculation.

The inoculation was performed at the growth stage V3 of each seedling with only two foliage leaves. The infection routes of the pathogen of bacterial blight of soybean include wound, stomata, and pores of leaves, and thus the inoculation was performed on the day when the pores were open. Greenhouse inoculation was performed to prevent a decrease in bacterial viability due to excessive temperature, a sufficient amount of water was supplied to provide proper humidity, and the inoculation was performed at 4 to 5 p.m. After the inoculation, the seedlings were placed in an isolation chamber for humidity treatment. Inoculum suspensions were sprayed onto both the front and back of leaves using a sprayer until water-soaking appeared (Groth and Braun, 1986). The density of inoculated strains was 8ra (106-107 cfu).

The samples were collected 3 hours, 12 hours, 24 hours, 3 days, and 10 days after the inoculation with the pathogen of bacterial blight of soybean (5 leaves per seedling).

### Example 2

### Extraction of Total RNA

Total RNAs for DNA chip analysis were extracted using TRIzol™ reagent (Invitrogen, USA) from the samples of a control group without the inoculation and the samples of experimental groups 3 hours, 12 hours, 1 day, 3 day, and 10 days after the inoculation with resistant strains (R46, R47, R48) and sensitive strains (S50, S51, S52). Quantitative and qualitative analysis was performed on the extracted total RNAs using Agilent's Bioanalyzer 2100 RNA nano kit (Agilent Technologies, USA), and the suitability of the DNA chip analysis was determined.

### Example 3

### Preparation and Analysis of DNA Chip (DNA Microarray)

Probes were designed for the preparation of a DNA microarray using Agilent eArray software (http://earray.chem.agilent.com/earray/) from 33,574 unigenes extracted from the Glycine max database at the National Center for Biotechnology Information (NCBI) of the National Institutes of Health (NIH), and a 60-mer oligonucleotide microarray was constructed (Agilent Technologies, USA).

The samples of the control group were labeled with Cy3 (green color), and the samples of each experimental group were labeled with Cy5 (red color), and the labeled samples were hybridized to the DNA chips prepared in a 2-channel array. At this time, the amplification of the total RNA was performed using a Low RNA Input Linear Amplification kit PLUS (Agilent Technologies, USA), and the chip analysis (including chip scanning) was performed using methods, kits and apparatus provided by Agilent.

For DNA chip data analysis, the calculation of signal intensity values of the genes, normalization (Lowess), clustering, and statistical analysis (ANOVA tests) were performed using Feature Extraction program and GeneSpring 7.3.1 software (Agilent Technologies, USA).

### Example 4

### Real-Time PCR

Messenger RNAs 17 genes resistant to bacterial blight of soybean, including AB052784 (SEQ ID NO: 1), AF022780 (SEQ ID NO: 2), BI944059 (SEQ ID NO: 3), U13987 (SEQ ID NO: 4), D86929 (SEQ ID NO: 5), BE823110 (SEQ ID NO: 6), FG999662 (SEQ ID NO: 7), CX710871 (SEQ ID NO: 8 or SEQ ID NO: 18), AF055369 (SEQ ID NO: 9), CS226295 (SEQ ID NO: 10), BU550410 (SEQ ID NO: 11), EV281281 (SEQ ID NO: 12), EV276436 (SEQ ID NO: 13), EF551167 (SEQ ID NO: 14), EH258681 (SEQ ID NO: 15), CX709057 (SEQ ID NO: 16), and CX702069 (SEQ ID NO: 17) examined in Example 3 were analyzed using a Bio-Rad iCycler system (Bio-Rad, Hercules, Calif., USA). The total RNAs were analyzed using an Omniscript RT kit (Qiagen), and the real-time PCR was performed using a SYBR supermix kit (Bio-Rad). All samples were amplified for 45 cycles at 95° C. for 20 second and at 60° C. for 1 minute. All data are shown as the means of three experiments±standard deviation (SD), and the primers used in the real-time PCR are as follows (Table 1):

TABLE 1

| | | SEQ |
|---|---|---|
| | | ID |
| Primer set | Sequence | NO |
| AB052784-F | ATCCGACAGACGCATTCTCA | 19 |
| AB052784-R | AGAGGAACATGCCAAAGCCT | 20 |
| AF022780-F | AGTCCAACCCAACCCTCAAG | 21 |
| AF022780-R | GCGTTGATGAGGATGCTGTC | 22 |
| BI944059-F | ACCACCCCAGATAATGAGGC | 23 |
| BI944059-R | GGTTGACACCAGCCATTTTG | 24 |
| U13987-F | AGTCCAACCCAACCCTCAAG | 25 |
| U13987-R | GCGTTGATGAGGATGCTGTC | 26 |
| D86929-F | TGAATCGCTGTATAGCCTCCC | 27 |
| D86929-R | GGAAACCTGTTCAGTGTGGC | 28 |
| BE823110-F | CAGCCACTTGAGGTGAAGGA | 29 |
| BE823110-R | GATTGTGGAAATGCAAGCCA | 30 |
| FG999662-F | GTGGAATGCTGATGACTGGG | 31 |
| FG999662-R | GCCTGCAGAGTCCAGTTCCT | 32 |
| CX710871-F | GCAGGTCTACCTCCAGCAACT | 33 |
| CX710871-R | AGTGGCTGCAGTCATTGCC | 34 |
| AF055369-F | AGTCCAACCCAACCCTCAAG | 35 |
| AF055369-R | GCGTTGATGAGGATGCTGTC | 36 |
| CS226295-F | AGCCTCAGCACCATCCTTTC | 37 |
| CS226295-R | TATGATAACCGGTGGCTTGG | 38 |
| BU550410-F | GGATCAAGACCGCGAGTACA | 39 |
| BU550410-R | CAGCCTCTTGAGGTCTTCTGG | 40 |
| EV281281-F | AGTGCCCTCAACCTGACCTC | 41 |
| EV281281-R | GACGAATGCATTTGGGACAG | 42 |
| EV276436-F | AGTCCATTGTCTTGGCCTCA | 43 |
| EV276436-R | CTAGCGCGCACACTTAAAGC | 44 |
| EF551167-F | AAGGATGATGATGCGGTGG | 45 |
| EF551167-R | GCATCGTCAAATTCCACGTC | 46 |
| EH258681-F | AGCAAATCCTCGTGACCCA | 47 |
| EH258681-R | GGTGGAGCCAAGTTAAGATCG | 48 |
| CX709057-F | AAATCATCAGCACCCAAACG | 49 |
| CX709057-R | ACGATTTGGTAGCATCACCG | 50 |
| CX702069-F | ATCATCAGGACTAGTTGCCTCAA | 51 |
| CX702069-R | GGTGGGGTGAATGGCAGTA | 52 |

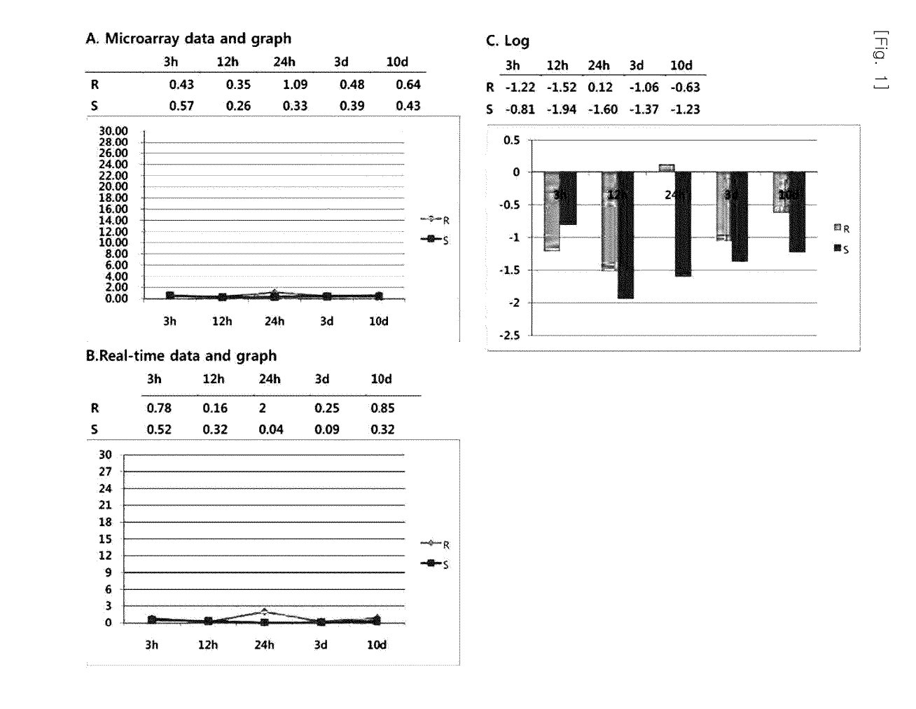

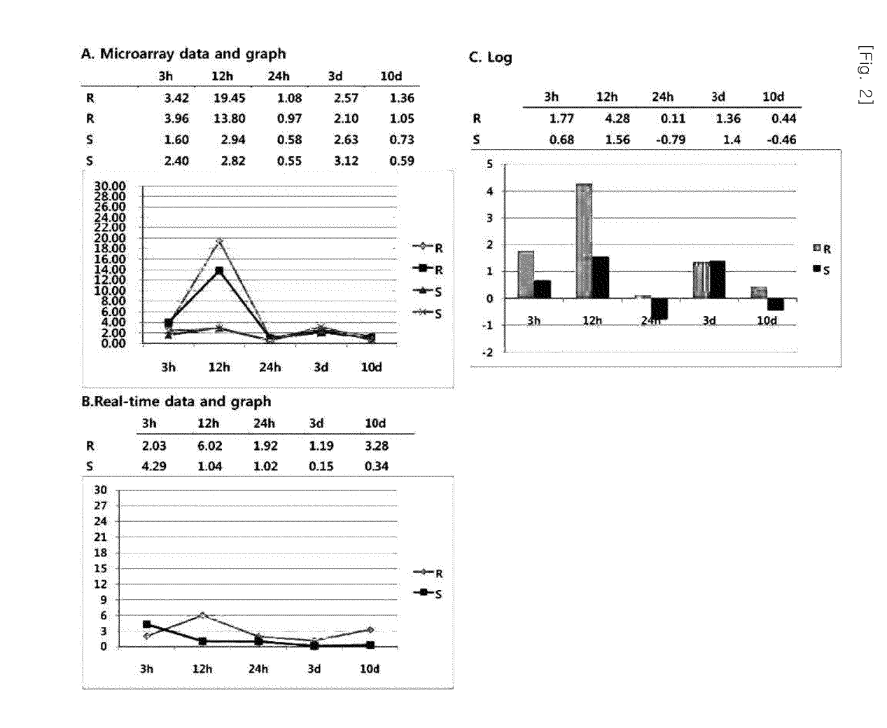

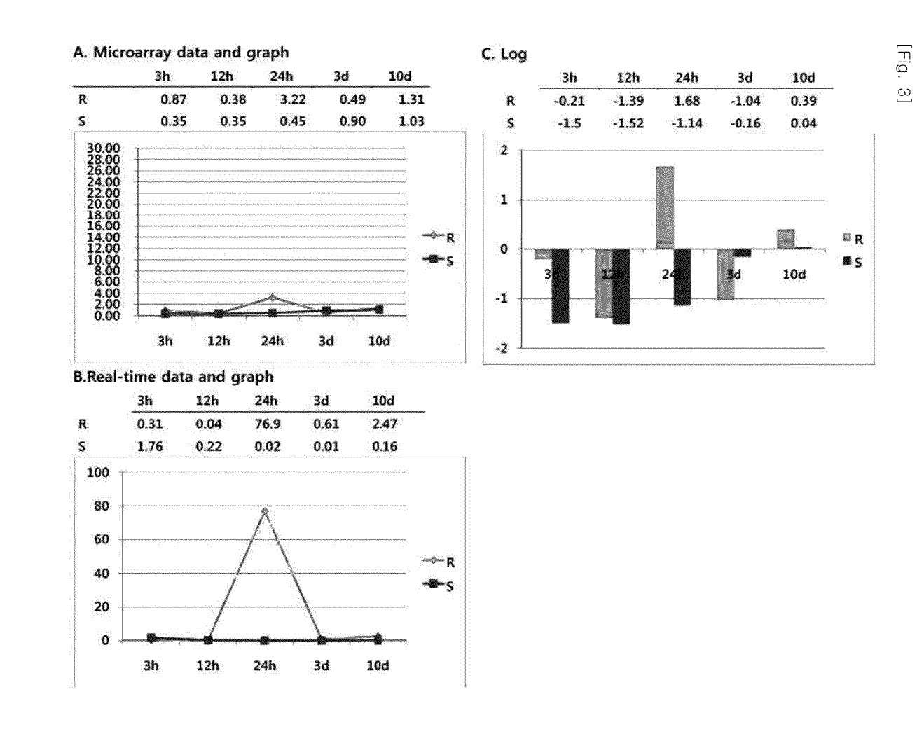

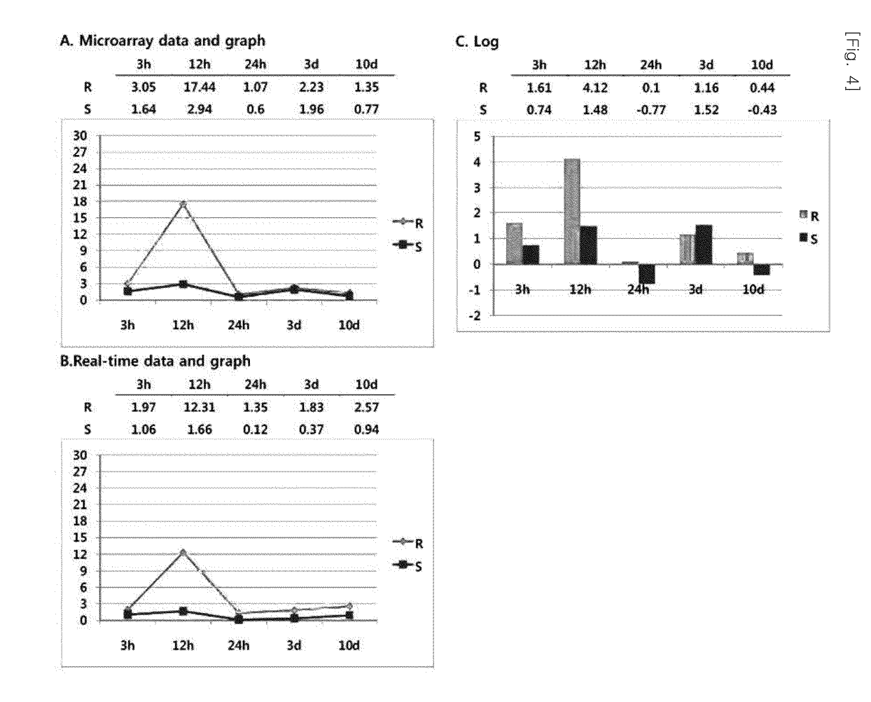

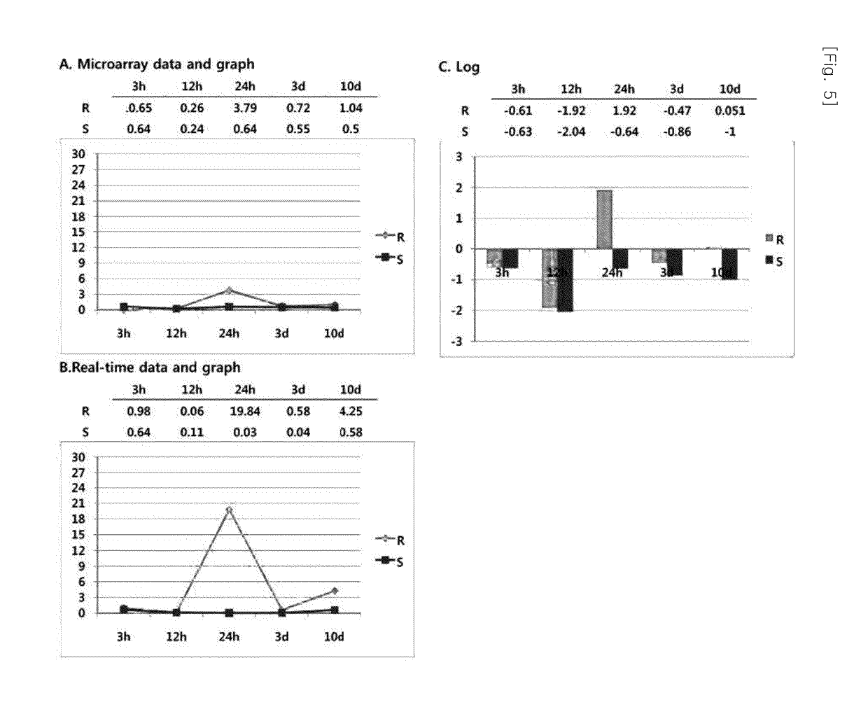

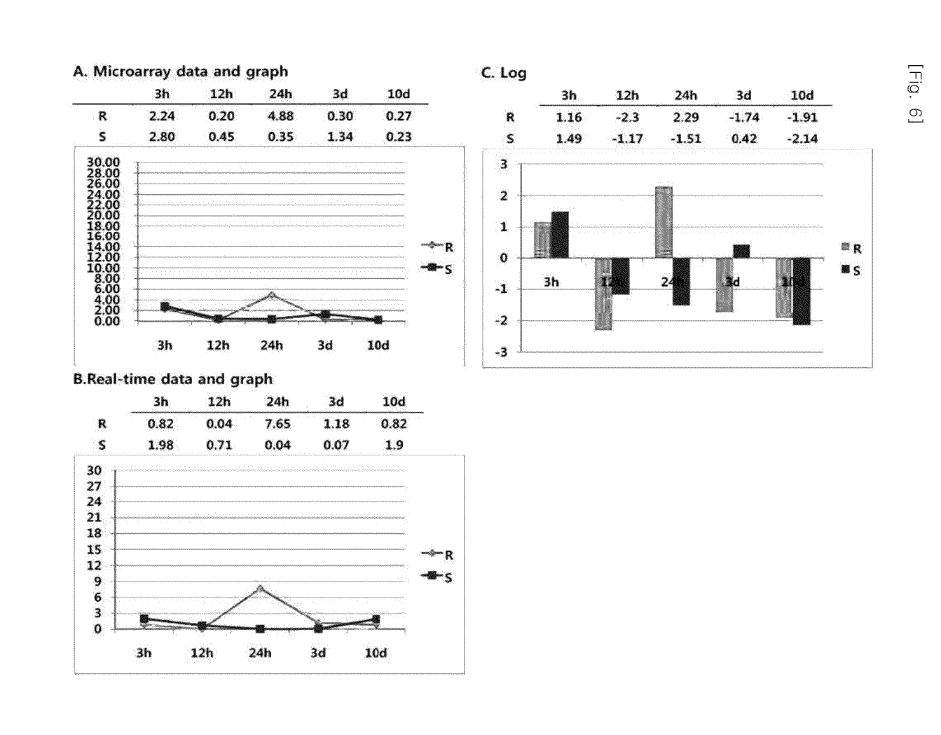

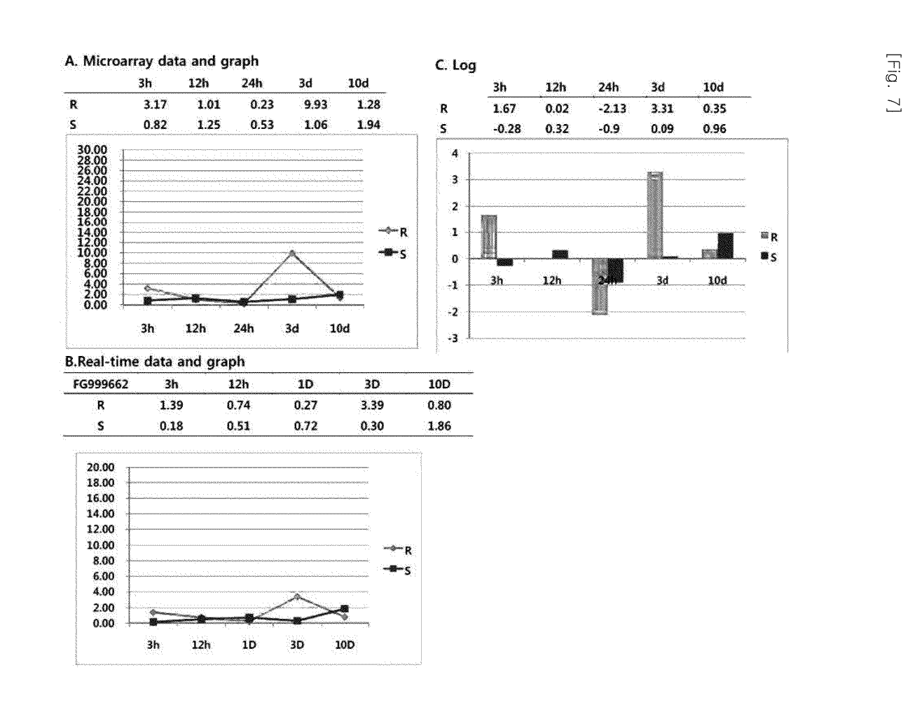

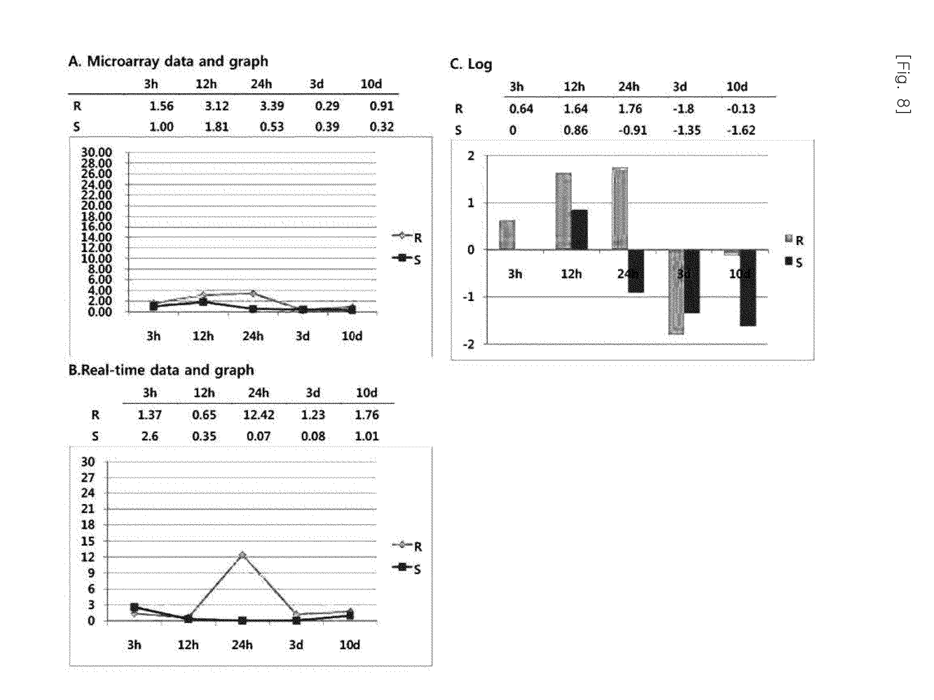

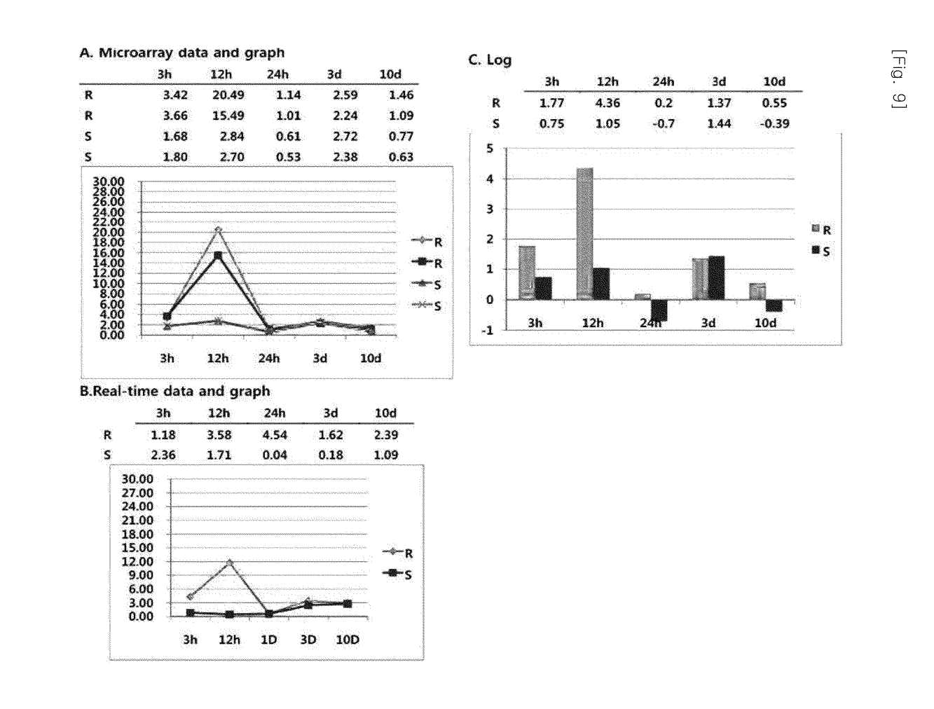

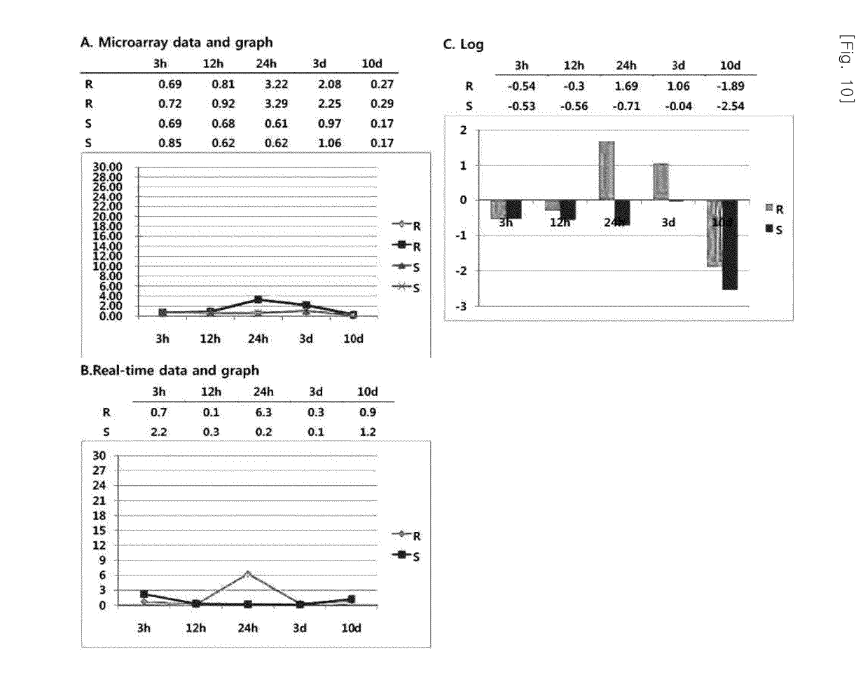

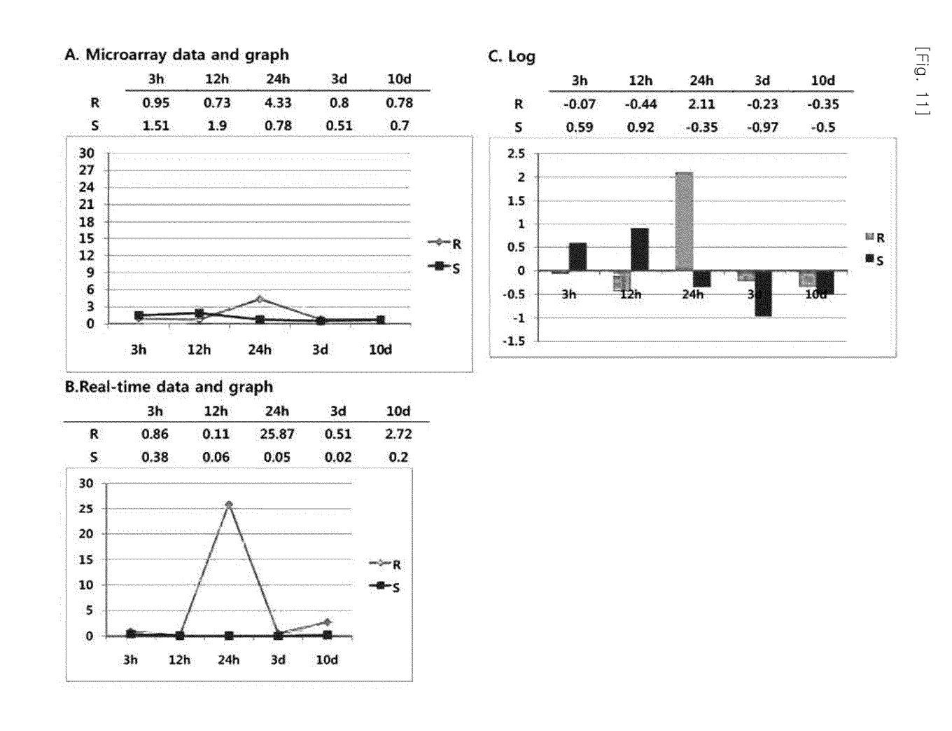

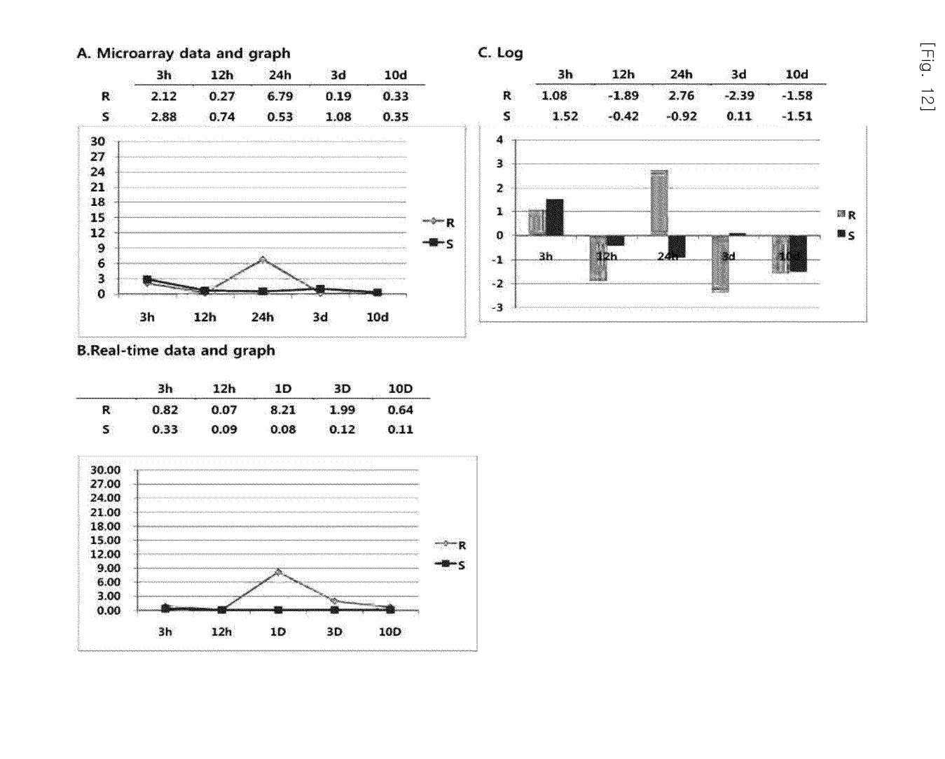

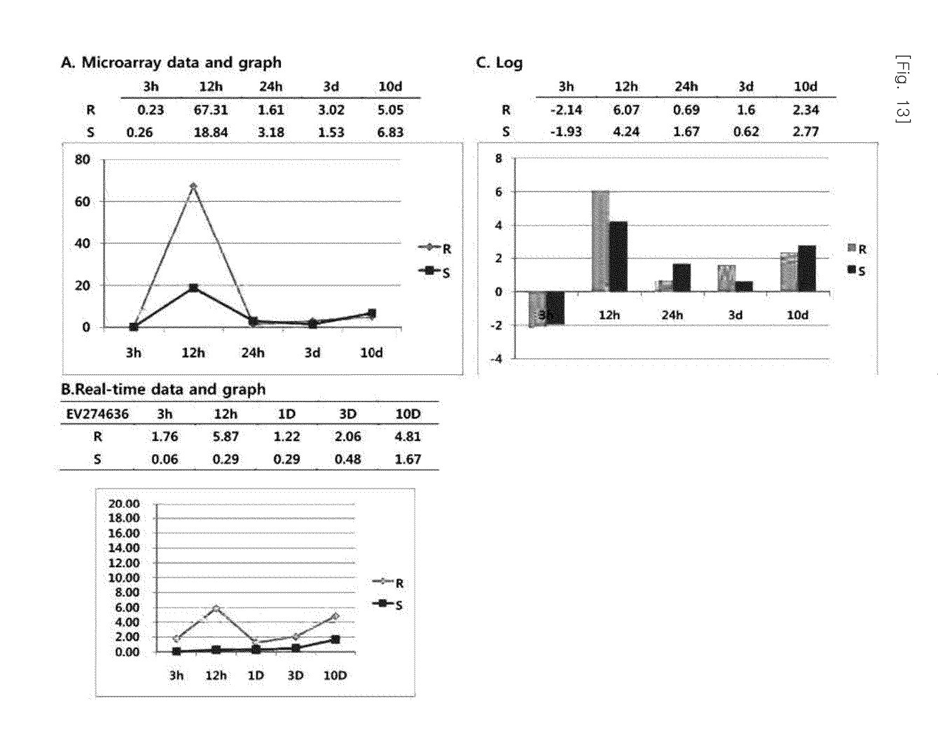

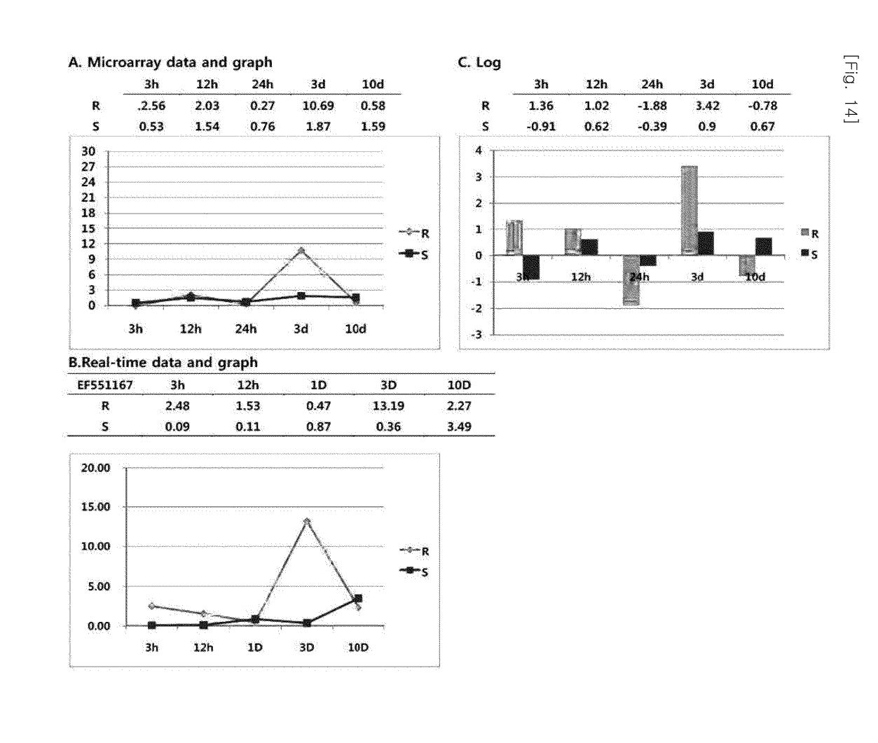

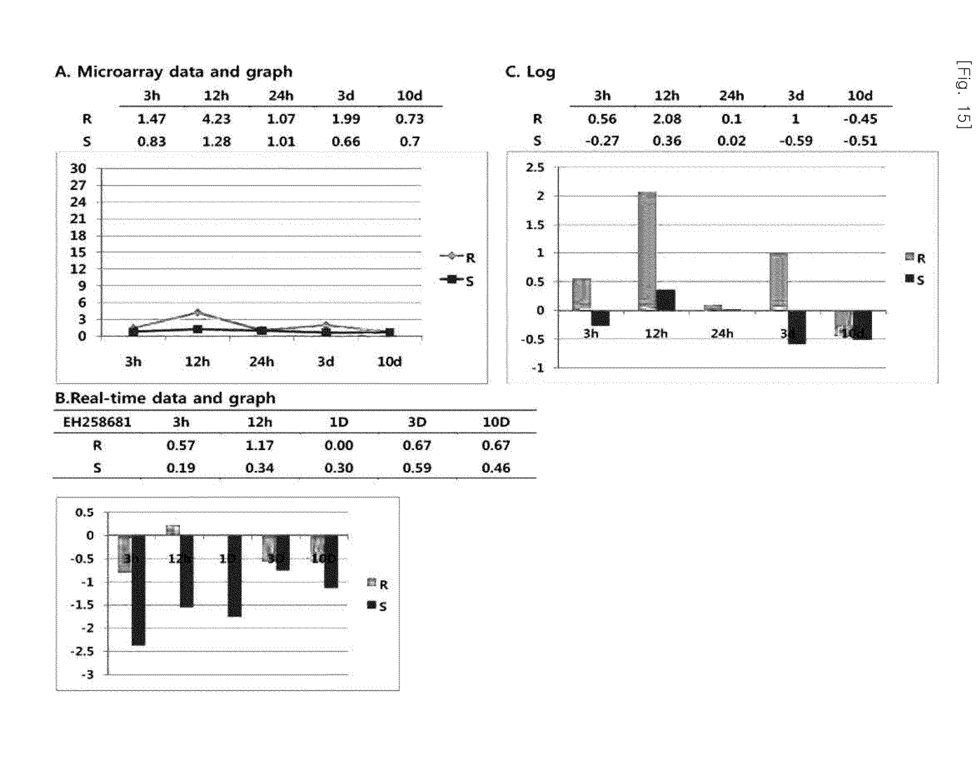

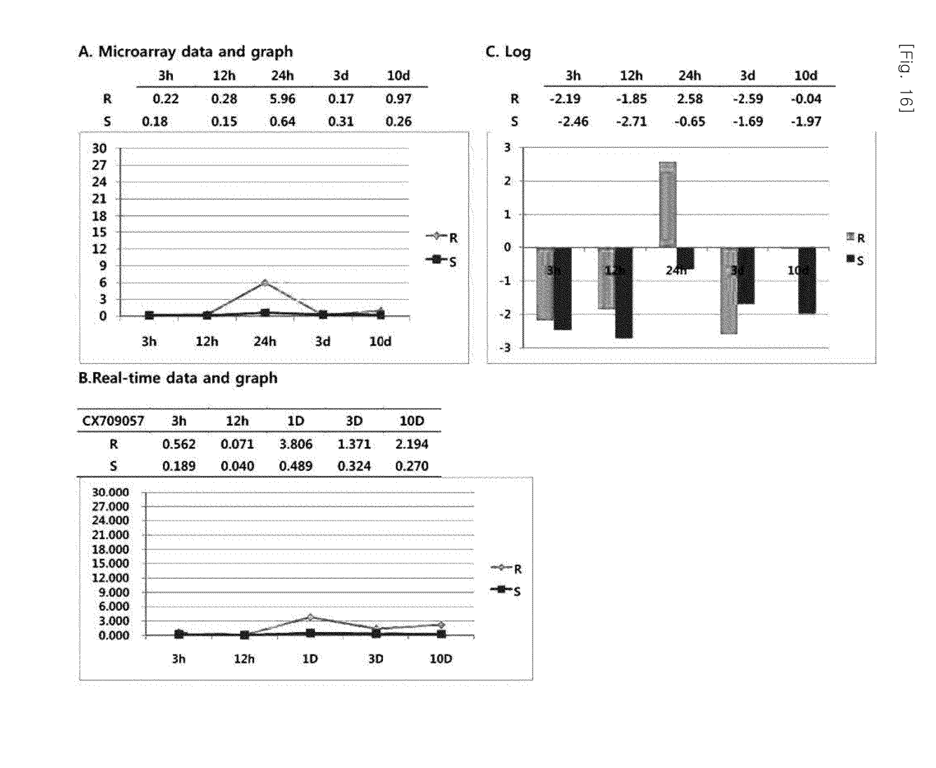

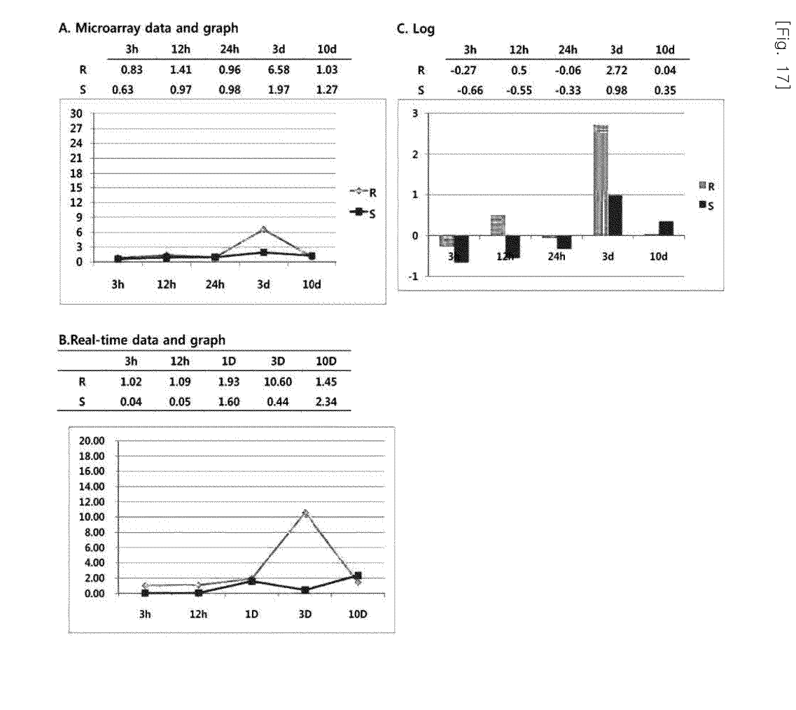

As a result of the experiments, the microarray data and the real-time PCR results analyzed in Examples 3 and 4 coincide with each other (FIGS. 1 to 17), from which it was found that the 17 genes are resistance to bacterial blight of soybean.

### Example 5

### Determination of Correlation Between Sucrose and Bacterial Blight of Soybean

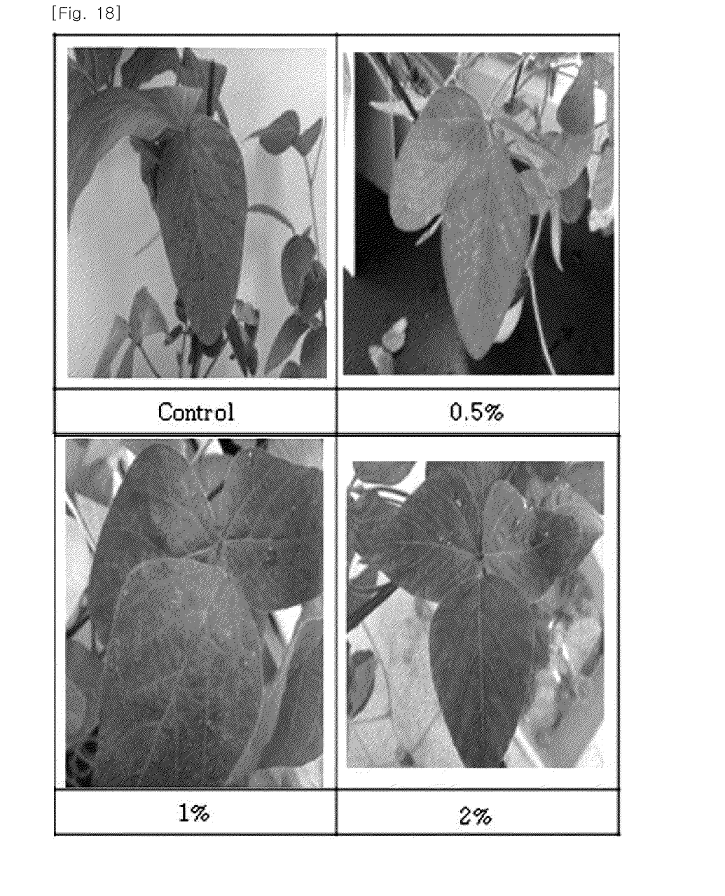

As a result of analyzing the functions of the 17 genes examined in Examples 3 and 4, there were a total of 13 genes associated with sugar metabolism. Based on this, the correlation between sucrose and bacterial blight of soybean was analyzed. As shown in FIG. 18, it was found that lesions appeared clearly in the control group untreated with sucrose, while the symptoms of bacterial blight were significantly reduced in the experimental groups treated with 0.5%, 1%, and 2% sucrose.

### INDUSTRIAL APPLICABILITY

As described above, with the use of the marker gene for diagnosing resistance to bacterial blight of soybean according to the present invention, it is possible to breed varieties that are resistant to bacterial blight of soybean, thus providing disease-resistant and high-quality, superior varieties.

1. A marker composition for diagnosing resistance to bacterial blight of soybean, comprising at least one gene selected from the group consisting of AB052784 (SEQ ID NO: 1), AF022780 (SEQ ID NO: 2), BI944059 (SEQ ID NO: 3), U13987 (SEQ ID NO: 4), D86929 (SEQ ID NO: 5), BE823110 (SEQ ID NO: 6), FG999662 (SEQ ID NO: 7), CX710871 (SEQ ID NO: 8 or 18), AF055369 (SEQ ID NO: 9), CS226295 (SEQ ID NO: 10), BU550410 (SEQ ID NO: 11), EV281281 (SEQ ID NO: 12), EV276436 (SEQ ID NO: 13), EF551167 (SEQ ID NO: 14), EH258681 (SEQ ID NO: 15), CX709057 (SEQ ID NO: 16), and CX702069 (SEQ ID NO: 17).

2. A composition for diagnosing resistance to bacterial blight of soybean, comprising a substance that measures the level of expression of the gene of claim 1.

3. The composition of claim 2, wherein the composition comprises at least one primer selected from the group consisting of SEQ ID NOs: 19 to 52.

4. A diagnostic kit for diagnosing resistance to bacterial blight of soybean, comprising the composition of claim 2.

5. A method for diagnosing resistance to bacterial blight of soybean, the method comprising the steps of:
isolating total RNA from a soybean sample;
amplifying a target sequence using the isolated total RNA as a template by performing an amplification reaction using at least one primer selected from the group consisting of SEQ ID NOs: 19 to 52; and
detecting the amplified product.


SEQUENCE LISTING (52 sequences)

SEQ ID NO: 1 - DNA, 1653 bp, Glycine max
atgcatccacctcactgtggaagtgagagcatcacatgcacaggcccaaccacagggcaaatgacatttctgcttgctggatttggacttctaattgtgggagctgcagggatcagaccttgtaacttagcatttggagttgatcagttcaaccccaacactgagtctggaaaaaagggaatcaacagcttcttcaattggtacttctttacctacacatttgctcaaatggtgtctttgtcactaattgtctacatacagtcaaacagcggagcgcagcgcagagaagcccacccagttaaagcaactggtccagctccactaacaagtttagctcaagctgtagttgtggctataaagaaaagaagactgaatctatcagaatatccactggattcttcgctctttgcctatgtttctccccagtctattaactccaagcttcttcacacgtctcagttcaggttcctagacaaagctgcaattataaccccacaagacggcataaacccagacgggtctgcatcagatccttggagtctttgcagcatgcaacaagtggaagagctaaaatgcttgctgagagtgatccccatttggttcgcaggaattttcttctacattgcaatagtccaacagaacacaatgctggtcttccaagcccttcaatccgacagacgcattctcagcaccaacttcaagatcctagcagcctcctacaccatcttccagatgctgagcctaaccatatggctacccatctacgaccgaatcctcgtgccgtcgctccaaagggtcaccaaaaaagagggtggcatcacagttcttcaaagaataggctttggcatgttcctctctatactatgcacaatggtgtctggtgtagtggaagaaagaagaaggacattggctttgaccaatcctattggattagagccaagaaaaggtgccatttcttcaatgtcaggtctatggttggttcctcagctaacactggcagggttatctgatgcattcgcgattgtgggacaagttgagtttttctacaaacagtttccagagaacatgaaaagccttgcagcgtctctatttttctgtggcctcgcgggatcaagttatttgagtagtttgctgatttctattatccatagggcgacagctaaatcttcaactgggaattggttacctcaggatctcaataaggggagattggattatttttattacattataactgctctagaagtcgttaatttcggttactttatattgtgtgccaagtggtataaatacaaagggactggttctagcagcagtggtgaccttcaactcgatcaagtatccaaaccatccgaaagaactgttaacactgtctagtggttctcatttaaacctgtggttatattaagtacttagctttaagttttgtggtttataatgatgttattttactgatcgtctttcatacagaatgtctgtaatcttaaacctaaaaaaatggttgagcttacggttcataatgtttctttagaaagaacaaataaggtagtctttctttatttttaggattggattggggaaatagttttattgtaaaccttcttgttttttttagtattgatagcttgcctttagtgaaaccaaaaaaatata

SEQ ID NO: 2 - DNA, 3133 bp, Glycine max
tcgttcaaaaacaaaaccattctaccttctccaaaaccatccaactctcatgttcgaagaaaacatacacaatctcttcaaactctaattttcttttcctttcatttaatttcctcaatggcggcttccgtcgaccagcgtccgtaccccggcctccacaatggcgtcgttcggcccctcaaaccaggcccggatatcccgcgtcccaagaagctaccccagccgccgccgccactctcggattcctcaagcgacgaagaagaagacaccaccttgaacttgaaggacctgattcgcaagggaacaacggaagttgaatcctccattttcgacccgcgcgacgatggaacctctgaccactggatccaacggaactcttcccttgtccgtttaacgggaaagcacccgtttaactcggagccacccctccctcgcctcatgcaccacggcttcatcaccccggtccccctccactacgtccgcaaccacgggccagtcccccgggcccgctgggaggactggaccgtggaagtcacgggcctggtcaccaggcccacatgcttcaccatggagcaactcctccacgatttccccagtcgcgagttcccagccacactcgtctgcgctgggaaccggcggaaggaacagaacatggtgaaacagagcatcggcttcaactggggggcagccgccatctccacttcggtgtggcgcggcgtgcccttgcgcaccctgctcaagagctgcgggatctacacccgcaccaagggcgcgctccacgtgtgcttcgaaggggccgaggatctgccaggtggaggtgggtccaagtacggaaccagtatcctgagggaggttgcactggatccctcccgcgacatcattctcgcctacatgcaaaacggggagcctttgtccccggatcacggcttccccgttagaatgataatacccggtttcattggtggccgcatggtcaaatggttgaagcgcatcattgttactaccgaccaatctcaaaactattaccattataaggataacagggtactcccttctcacgttgatgccgaacttgccaatgctcaagcttggtggtacaagccggactatattatcaacgagcttaacataaactccgtgataaccacgccttgccacgaggagatcttgcccatcaactcatggactactcagatgccttatttcatcagaggctatgcgtattccggtggtgggagaaaggtgacacgtgttgaggtaaccctggacggaggtgaaacgtggcaagtgtgcacactggactgtccggagaaacccaacaaatatggaaagtactggtgttggtgtttttggtctgtggaggttgaggttttggacctgctcggagccagggagattgcggtgcgtgcttgggacgaggccctcaacacccaacctgaaaagctcatttggaatgttatgggcatgatgaacaactgctggttcagagtgaaaaccaatgtgtgcaggccccacaagggtgagataggtatagtgtttgaacaccccacccaaccaggcaaccaatccggcggatggatggcaaaagaaaaacacctagagaaatcatctgagtccaacccaaccctcaagaagagtgtctcctccccattcatgaacaccacctcaaaaacctataccatgtctgaagtcagaaggcacaacaacgccgattcggcttggatcatagtccacggtcatgtctatgattgcacccgcttcctcaaagaccatcccggtggcacagacagcatcctcatcaacgccggcaccgactgcaccgaggagttcgaagccatccactccgacaaagccaagcaaatgcttgaagactaccgaatcggcgagctcaccaccacctgctacaactctgattcttcatcgtcaaacccctccgtgcatggcagctccgacaccatccctttgacccccatcaaggaagtgattacaccaatgcgaagcgtggctcttaacccacgcgagaaaatcccatgcaagctcatctccaaaacctccatctctcacgacgttaggcttttccgctttgccctcccctccgacgacctactcatgggtttgccagttgggaagcacatattcctatgtgccaccgttgacgagaaactatgcatgcgagcctacactccaacaagcagcgttcacgaagtgggatacttcgacctcgtcgttaaggtttacttcaaaggggtgcaccctaagttccccaacggtgggatcatgtctcaacacttggactctctccccattggttctgtcttggacgtgaaaggcccacttggccacattgaatacactggcagaggaaacttcctcgttcatgggaaaccaaggtttgccacgaggctagccatgttggctggtggcacaggaatcacacccatttaccaagtggttcaagccattctaaaggatccagaggactgcaccgagatgcatgtggtttacgcgaaccgaaccgaggatgatatattgctgaaggaagagcttgatgagtgggctaagaagtatgataggttgaaggtgtggtacgtgatacaagaaagcataagagaagggtgggaatacagtgtgggattcatcactgaaagtatcctgacggagcatattccaaatgcatctcctgataccttggcattgacttgtggaccaccacctatgattcaatttgcagtccagccgaatttggagaagttgggctacgacacccagaataacttgctcgtgttttaggggactaaactagaatacattgtatgtgtatatagtagtttattgttatatataaataatggattctattagctgcttcgtttcaagatttttttatgtttcaatttttaaataaaagtgtacatgggacagctagctagctacgcaggtttacttttcctcgtcaacttttgatgtagacaagtatgtcaattattttgactacttttgcatgtataaataaataaataaaacagtgtgcaactatcactttttcaatttatttactctgaatcaaaggcattacaacattatgtcattaatttaatgacattttatatgaataaaatacatattacaaggc

SEQ ID NO: 3 - DNA, 689 bp, Glycine max
tcaacagagaaacatttcactaatagagagacataagcataatattgagaaataatggagggttgtacttattgtgtgaagttgctatgccttgttgggtttgtggtgcttgtgagtactattgcaggggtggagagtgctggtgaatgtgggaaatctaccaccccagataatgaggcttataagcttgcaccatgtgcctcagcagcacaagatgagaatgctagtgtttctcagagttgctgtgctcaggtgaagaaaattgggcagaacccaagctgcctctgtgcagttctgctctccaacacagccaaaatggctggtgtcaaccctcaagttgctgtcaccatccccaagcgttgcaacctggctaatcgccctgttggttacaagtgtggaccttacactctgccttgaaagagttgaggttgttgctgaaaacaaagatgacagggcgtaagtgcactatggctgttggctactacaaactgtgactacatgtttgttgtatcgtcttgtaactttctagtgaataagagctagctgctcactagtgtaccctataataaaacatgtactttcggtgtttcatgtgcctactcaaacatttaactgttttaagttttaacaaaatcttttgatcatcgttacccaattaagtttgttataatatttaatattatctatta

SEQ ID NO: 4 - DNA, 3133 bp, Glycine max; identical to SEQ ID NO: 2

SEQ ID NO: 5 - DNA, 1284 bp, Glycine max
aaatcactcacacactctttcttaagttctcagaaaccttaaaaacaacaacgtaggagtgttcggaaaagatggctaagcaggaagtggtggaagggttcaagttcgagcagaggcacgggaaagaacgcgtgagagtggcgcgcgtgtggaagacgaggcaggggcagcacttcattgtggagtggcgcgtggggatcactctcttttcggattgcgtcaactcgtacctccgcgatgacaactctgacatcgttgctactgataccatgaaaaacaccgtgtatgcaaaagcaaaggaatgctctgacatactttctgccgaggagtttgctattctgcttgctaagcactttgtatcattttaccagaaggttactggtgctattgtgaatattgtggaaaaaccatgggagcgtgtcactgtggatggtcaacctcatgaacatggtttcaaacttgggtctgagaagcatacaacagaggcgatagtacaaaagtctggttcacttcagttgacttctggtattgaaggattgtcagtgttgaagacaacccagtctggttttgtgaatttcataagagacaagtacacagcacttcctgatacccgtgaaaggatggtagcaacagaagtaaccgcactgtggaggtattcgtatgaatcgctgtatagcctccctcagaagccgctttactttacagaaaagtatcaggaagtgaaaaaagttctggctgacactttttttggcccaccaaaagggggagtctatagcccatctgttcaaaacacactctacctgatggcaaaggccacactgaacaggtttcctgacatagcttatgtcagtctaaagttgccaaatcttcatttcatacctgtcaatatctcaaaccaggatggccctattgtgaagtttgaggatgatgtgtacttgccaacggatgagccacatgggtcaattcaagctagtttgagccgcctttggtcaaagctgtagaaactgcagaaactatggtgctcatcccttctggtggattgctagtgcagcctaaactagcaaacctggcctcagataatgctttccattaatgtgtcttgtaattctggtttttgttatctttatcttttgttttgtttcatgaagaactattatgcatgaataacttccaggagggatattctgtgggttgcaccaatgttaagctttttgtttctactattccaataatttcctagtgtccttgtggaatctgaaataagtcttactatgacgttgatgc

SEQ ID NO: 6 - DNA, 1355 bp, Glycine max
ctacaactaaacacactagctatagctagaatcccctccaatccctctttgtctttgtgcttttctttagttctttccaaaacagcaacaatgatgagttgccaagcatggcctgaacccatagttcgagtccaatccttagctgacagtggcctaagctcaatcccttcacgctatattaggccacactcccaaagaccctccaacaccacttcttttaaactttcacaaaccgaacatgatcatgaaaagatattccgacacgtggacgaggcgtgtcgggagtgggggttcttccaggtggtgaaccatggggttagccatgagttgatgaaaagttctagggaactgtggcgcgagttcttcaaccagccacttgaggtgaaggaggagtatgccaactcccccaccacttatgagggctatggaagtcgcttgggagtgcagaaaggtgccaccttggactggagtgactacttctttctccattacaggcctccctcacttaggaaccaagctaagtggcttgcatttccacaatcctttaggaaagtgattgctgaatatggggaggaagtggttaagctaggaggaaggatactcaaaatgatgtcaataacgggatcctctagagattccctctcaatgcatttgggggaggagagtgaggttggtgcatgcctaagggtgaatttctacccaaagtgccctcaacctgacctcacttttggactctcccctcactcagaccctggtggcatgaccattcttctctctgatgactttgtgtctggccttcaagtaagaagaggagatgaatgggtcatagttaagcctgtcccaaatgcattcgtcatcaacattggtgaccaaattcaggtgctgagcaatgcaatatacaagagtgtagagcacagggtgattgtgaactcaaacaaagatcgtgtgtccttggcattgttctataacccgagaagtgatttactcattcaacctgccaaggagcttgtgacagaggagaggccagccttgtactcaccaatgacctatgatgaatacagactttacattaggctaaatggaccttgtggaaaggcccaagttgaatcactggctttccaaccatgacttaataatgtcaattaataacgtgtttggtttcacaatgtaattttctatgtttcatgtgtaattttaagttgatagttataacttatatttcccacataattttgtacttcttaagagttttcaaacatgttattacctttcctttaaaagtattgatgtttaaagtcaatgtatagatattaagaaataactatttaataatacccttaacttattatgtgaca

SEQ ID NO: 7 - DNA, 1101 bp, Glycine max
  modified_base (112)..(327): a, c, t, g, unknown or other
ataacattatccattcactaatcataatcacttccaattgctctcactagtcactactactactggagatggcttcaactgcctcttgcacttctatttccccttctagtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttatcttcaaaaggatctacttgggatgagatagactatgagtttcttggaaacttgagtggtgatccatacatccttcacacaaacgttttcagccaaggcaaaggggacagggaacaacaattctatctgtggtttgaccccactgctgatttccacacttactctatcacgtggaacccccagaggattatattctctgttgatggcactcccattagagagttcaagaacttagagcctattggggtccccttcccaaagaatcagccaatgaggatgtactctagtttgtggaatgctgatgactgggccacaagaggtggccttgtgaagacagattggacacaagctccattcacagcttcctacagaaacttcaatgctgagacttgcatatggtcttctggagcatcttcttgtgctaaaacttcttctacttccaccactagcagtggttcatggctttcacaggaactggactctgcaggccatcagaggttgaagtgggtgcagaagaactacatgatttacaattattgcacagacactaagcgtttcccacagggccttcctccagaatgcaacatctctggagagaaaaacaaagcaacgcattcctcttatattttctatgatcagaatgtcataccaatattagtttaattcttgatgtagctattgtagtttaattctttcatttgttcccctgtttgtcaccactgtgttaacaaatacggccatttttattataaaatagattcttttcaatag

SEQ ID NO: 8 - DNA, 1596 bp, Glycine max
gagttgcataggtcaaatgcagtagtttattgaaacagattccctttgacccgtgacattttaagtggtgactactagaccaatggaatggagtgcaaagaacatttcagttacctctgctaaatgggtcactactactctgtctctgctctccctgttttggctcttgtccctgcaccaagcaagagctgcaaagttccacaatgtgagtggcttgagagggaaaaagccagttgtagcaaatgggtgcaatttgttcctgggaagttgggtggttgacacgtcttatcctctctatgactcttcaacatgccccttcattgatcccgagtttgattgccaaaagtatggaagacctgacaaacagtacctcaaatacgcttggaaacctgattcatgtgccatacccaggttcgatggtgcggctttcctgaacagttggaggggtaagaagataatgtttgtgggtgactcactgagtctcaacatgtgggaatcactgtcctgtatgattcacgcgtcggtgccaaatgccaagaccggctttttgagaaaagaatcactgtccactgtgaccttccaggactatggagtaactatacagctataccgcacaccgtatttggtagacataattcgtgaaaatgtggggcgagtgcttacattggactccattgttgctggaaatgcgtggaaaggcatggacatgctgattttcaactcgtggcattggtggacccacactggaaaatctcaggggtgggattatattagagatggacccaatcttgtgaagaacatggaccgtttggaggcctacaataaaggattaaccacttgggctaattgggttgatcagaatgttgatccctccaaaactaaagtcttctttcaaggcatttctcctacacactatcagggcaaggattggaatcaaccaaaaagaagctgtagtggagagcttcaaccattgtctggatcaacatatccagcaggtctacctccagcaactaccatattgaacaatgtgttaaggaagatgagtactccagtttatctacttgatataacactcctctcacaattaaggaaggatgctcacccttctgcttatagtggggaccatgcaggcaatgactgcagccactggtgcctaccaggattaccggacacttggaaccagctcctatatgcagctctcaccaaatgaaggtgaaggccccatacaaattgatccttttcttcatcccttggttatatattcagctagtgtttttagaatttctttattatttgggggatatatagatactacagaggggtgaaactcaattgtaaaattatatataggattcttggaagaataatttacaaaacaaaataagttccttagagatcaaaattgttgcatactaagatgagtactttgtttacaagggttcgtaaactaattttattgaatcacagaatgatgttgaattctatcattggttgatgcacggatcatgtataacatcccatacttgtgacagctacagggttaatcgtaactatgaaactgtttccttccttctgagaaagaaagggtcttattgc

SEQ ID NO: 9 - DNA, 3133 bp, Glycine max; identical to SEQ ID NO: 2

SEQ ID NO: 10 - DNA, 1305 bp, Glycine max
  modified_base (911)..(933): a, c, t, g, unknown or other
cttgatttttttacaataacccaatcatagtctcactcgggattggttattcttattctaagagataatccaaatccaaacccaaaagaacaacacaatgaaaggagaatacgtagagcaacagcaacaacatcctaagagtgaaacaccacctagtatgttctcaaagcttcagcctcagcaccatcctttcccgcaccaccctttccaactctccgccgaggatgccaccaccatcaccccgagcaccgctcagaaggccaactcctccggcggcgacggcgcgaccatcgaggttgttcggcgtcccaggggacgtccgcccgggtccaaaaacaagcccaagccaccggttatcataacccgtgacccggaacccgctatgagtccctacattttagaagtgtctggcggaaacgacgtcgtcgaggccatcgcgcagttcagtcaccgcaagaacatgggaatctgcgtcctcacgggttctggaaccgtcgctaacgtcacgctccgtcaaccttccaccacgcccggcaccaccgttacttttcacggccgtttcgacatcctttctgtttccgccaccttccttccgcagcagtccggcgcttctccggcggttcccaacggattcgccatctcgctcgccggaccgcaggggcagatcgtcggaggacttgttgccggcgggctgatggctgccgggactgtttttgtgatcgctgcctcgttcaacaacccggcataccacaggcttccgccggaggaagagggcgcctccgccggagatggacattcgccgccagtctccggcggcggagacagcgggcatggacaggcggagtcgtgtggaatgtccatgtacagctgtcacttgccctctgatgtgatttgggctccaacggctagannnnnnnnnnnnnnnnnnnnnnnttactgatcgatcaatcacacgtcgagtttctgtttctttttcttctgaggttaactttgtggttattgtaatcaattctactatatgttgctctgtttaatttgatttagttggtgtagttgatatatctggtattagtttaannnnnnnnnnnnnnnnnnnnnnnnnnttaagtttataaatggaaacattagtatagtatagttattatgtagctagggtggtttatttgcggcgctaactgtattatgtcgatggctctctaccttgttgaagagattttgtttatcgaccattatgactagtgagcttgttgtttattttagatttaaaatttttgtaatggattttataataatgataatgagtaa

SEQ ID NO: 11 - DNA, 2142 bp, Glycine max
  modified_base (63)..(87): a, c, t, g, unknown or other
tcaccctctcacctcgttcttccaacccctctagatttgaatcaccaaacttcctcgttcctnnnnnnnnnnnnnnnnnnnnnnnnntatatttgtactgtacccaaaaaagcacttcacaaagttgaaagctgaaacctcttcttcaatctacttcacgatggcggagaagcacttgcgggagctccttaaagaggatcaagaacccttccttctgaaacactacatttgggagcgtcgcaggcagctcaaaagaccttccccaaacagccctttacaacaacaactgaaaaagaaaaactcaaacttcccacttaacaaatgcttcctcacttccctacaaaacgccacaaaatccccactcttattgtctccaaagaatagtacttctcccctcctcagaccctccaacgccaaaaccgcgtcgctcctcctcgaagccgccctcagaatccacaacaaaaacgcgaaaccgaagaagcccaaaccaaacagcgccttgggcatcttcggctcgctcttcaagaaactaaccaaccgaaagagggaaacattagagggtgttcttgttaaggttaaagacagcgacgacgatgttgttgtggtgggatcctgtgaggtcgggttcagttgttcctgcaacggaaggccaagcagtgctgtgtggtcggagagcaacgaggataaatcgttggacttggaatcttctagtagtggccactcatttgatgactctgttgctgaagagattgagtttctcaacaaacgcaaacatgttacggattctgcttgttttgacgacgatgatgatcacggcttcttctgtgaaagcccgttccgtttctccctccaacgcagccccggctactccggccgccacaccccggatttttcttcgccggctgcgtctccatgtcgccgcaaaactgaggacaaaaaaattaatggtgctgatggcgtcaacaaattccaatcaggggaagaagaggaagataaggaacagtgcagtccagtatctgttttagacccacccttcnnnnnnnnnnnnnnnnnnnnnnnnnnnnnnnnnnnnnnnnnnnnnnnnnnnttggactgtagctatgccaatatgttttgtgctaatgccacctatctttggtgggttccattggaagattgcaatagctcatcatccaacatgcatttcgcattgaatgtgaaattggactactctttgttcagagctataaatttcaacatgacccacaaattttttgtcaggagaactaagcagcatctactaaacagacttcgtagattcgagaaacttgccgagttagatccggtagaacttgagaagagaatgctggatcaagaccgcgagtacaaaacatttactgaagaggacgatgattgtgaagacggcgacagcgaaacagcatgcaaagaaaatgctctgagaggagaattaatgtttgaaatcctctgccattcaagtgttgaagacacacaacagcaaactccagaagacctcaagaggctggtctatgatctcatcaaggaggaagagagggaagttaataattcttcagacatggtgataagaagggttcttaggaggctagaattgtggaaagaagtggagtccaacactattgatatgatgatagaagaggacttctctagagagcaatgcaggtggaagaaaaatgctgagcacacaagtgagctagctgtggaacttgaacttgctatcttttgctttctagtggaggagctttcagaggaactagtgtgttaatgtgggcaaattttgatgcagctgaagaatttagtatgagttatttattatctttattcttttaattagaagagtacagtctaacttgcaccttatttaaataggctagttagataaaatgagacccaaaaggataatttgggttggaggagggagaacttgagggggggatatgttacatatgtacatgactcgtgtatgtaggtgccagggaatgctgtaaatcacctttattagttacaaaatactataatttttgcagtcattgatcccccgggcgttgttataatgctatgttctatttaatgtatccacatttgtaattagcattggtatctatatatcattcatatttatcttta

SEQ ID NO: 12 - DNA, 1355 bp, Glycine max; identical to SEQ ID NO: 6

SEQ ID NO: 13 - DNA, 2440 bp, Glycine max
atgcactcccataacaagattagtactttcgaattctccaccacgcttaaaagacagccacaaataataatttttgttttcttattaaggaccgcgatacgaatagcgatagtgcaccagaagctcatcagatacgagaggagagagagaatgggagaggaagaagaaggtgcgagacaacaagagtctcatgttgatgttagtttgtttgagacaaaagaagcaagattccgaggagtttacaaggtatttgcatctacaatatttgcagcaatatgtttgatttgggtgtaccgagtggcaaacatccccacagttgcaagtgggagatggacttggattagtgttatggtgtcagagttatgttttggaatatactggatcatcactcaatctgttcgttggaaaatcgtatatcaaacgcctttcaaacacaggctcttacaaaggttagtttctcttcatcacaagcctcaaaataaagagagagagcctttattttattttttttatccattgcaccatatataccaatttttacctccaactttccatatttcttttgttgagttgagtcttgcaaatatgatgaggagagtttaccagcagtggacatttttgtgtgcacggctggacccaacacttgagccaccatgcatggtgatgaatacgaatttgtacaaagacatgaaaagtgagattgaatcagccgtagcaaggggcgaggttccagataatgcgatgaatcagcacagaggattctcagaatggaatccaaaaataacaaagcagaatcatcaacctatcgtgcagataataattgatggaagagacactaatgctgtagatgaagacggatttcaactgccaagagtcgtatacatggcacgcgaaaaacgacacaactaccctcaccacttcaaagctggagcggttaatgcattgatacgagtgtcttccgagataagcaacgcccctttcattctcaacttggattgtgatatgtactccaataatgcaaatacaattcaagaagtattatgcttcttcttggatgaaacaaaaggccacgatatagcttatgttcaatttccacagagctataataacatcgccaagaatgaccattatgcaaattcttatcttgtgagcaataagtttgagctagctggaatatgtggatatggagcagccttgttttgtggaactgggtgtttacatcgaagagaaagtctttcgggagcttacttgaaagattacaaagcaaaatgggacagcaagccaaaaagaaacgataatagaacaattgatgaattgaatgaagcatccaaggttcttgccacttgcacctatgaggagagcactcaatgggggaaggagaagggattagtatatggtattcccgtggaagacattgctactggccttgtaatctcatgcaggggttggaaatcgatttattacaaccctgaaaggaaggctttcatgggcatagctccaacaaccttggatgtagcatgtcttcaacatatgaggtggtcagaaggcatgttccaagttttcttctccaggtattgccctttcatttatgggcatgggaagatacactttggtgtacaaatgggatattgcacttacctcttatgggctcccatgtctctgcctactctctgttatgtcattgtttctcctatttgcctacttcatggcattccgctgttcccacagctttcaagcatatgggtccttccatttgcgtatgcatttcttgctacatatggctttagcttgtgtgagtatttgatttgtgggtccacagcaaagggttggtggaacttgcaaagaattaaattcatccataggaccacctcgtatctctttggtttcattgacaccatgaaaaagcaattaggactatcacaaacaaattttgtcatcaccaataaagtggttactgaggatgtccaaaagaggtatgagcaagagatcattgaatttggaggctcttcgatcatgttaaccatgttagcaacagtggcattgctaaaccttgttggtcttgttgggggaatcaagaggattatgatggatttaaatttggaattcagctctagccaattgatgatgcagattacactgagttcattggtggttatgatcagtttaccagtgtatgaggcacttttcattcgtagtgataaaggttgcataccatcttcagttatgctcaagtccattgtcttggcctcattagcatgttgcctggcacctttcatttgctaatctagctatttaaatgtcagtacaatactgaaagtgtggctcattacttactagtgataagaatctttaatattagttcctagtatggcttcattgacttgaagctttaagtgtgcgcgctagttatctttatcaaagtactttggttccacgtatc

SEQ ID NO: 14 - DNA, 814 bp, Glycine max
gcacaacgttagttactactaactaccccttttgcacacgttgtctcattcactcaaacaacttcaaagaacgaagcttttcaccaagtaacatgttttccatcaatcatttctccgacccagatgccacctcgccggggtcgggaggaagctcccgaccggcgctgtcggacgaggatttttttctggcggcaagcaatcccaagaaacgcgccgggaggaagaagttccgggagacgcgccacccggtgtaccgcggcgtgagaaggcgggactccggcaagtgggtgtgcgaggtgcgcgagcccaacaagaagtccaggatttggctcgggacctttcccacggcggagatggcggcgcgtgcgcatgacgtggcggcgatcgcgctgaggggaaggtcagcctgcctcaacttcgccgactccgcgtctcggctcccggtgccggcgacggcggaggccagggacattcagaaggctgcggcggaggccgcagaggcgtttcgccctgggaaggatgatgatgcggtggcggagagggtggcagcgacggcgacggagcgtgaagaagaacaagaagaagggatggtgccggagtatctgaggaacatggtgctcatgtcgccgacgcattgcttcgggagtgatgagtatggaagtgctgacgtggaatttgacgatgctgaagtttctctgtggagttactccatttaatttaactcagttattggattttttttttactattttgagtaatgtaaagattgttcgtatgggacaagtagaatagtgtccgtacttataatttttta

SEQ ID NO: 15 - DNA, 1014 bp, Glycine max
  modified_base (433)..(454): a, c, t, g, unknown or other
aacaccaccacgtaactgcgtgcactctctctctctctacaacaagaattcatctattcatttcagcccatcagcatggcgaaggagaagaaaaacttgtccaacaagaacaagaagccaagcaatgcagacgaggttcacttccgcggagtccggaagcgcccctggggccgctatgccgccgagatccgcgaccctggaaagaagacccgcgtctggctcggcaccttcgacaccgccgaggatgccgcccgcgcctacgacgtcgccgcccgaaacttccgtggccccaaagccaaaacaacttccccttcctccccgacaacaacaccaacgttaacctcaacaagaagaacaacaacgttaacgttaaatctgacagtcccagccagagcagcaccgtggagtcctccaccccggagcgtgaattcannnnnnnnnnnnnnnnnnnnnnaatggatcgctttccgttcctacccatccagcagcaaatcctcgtgacccaccccgtcgcacccatgcgcccggtttactttctcgaccgggcccacttcatgacccagcccttcccactccgattcgaaccgggtccggtccagagcgactccgactcgtcctccgtcgtcgtcgattgccagcccaaaagagagctcaatctcgatcttaacttggctccacctaatgaatattaacctttttttttaatcttttaatttttcataaaaaagacagcagaaagaaaggaaaggactaaggagannnnnnnnnnnnnnnnnnnnnnnnnggaagattaatcttccattaaattgtcgtgtagattagataagatgttgttaattacatcctttgatgtaaataggttaatttttctgtttttttaggtccttgatagtattaacaaaggacatgatctgaaattgtctagatctggtataattttcatgtaacatcggaatatctgatgtaattctggctaatgttaattttctcttattacaataatttctgctattg

SEQ ID NO: 16 - DNA, 2203 bp, Glycine max
gattaaaccaatactcataattcaaacacatattatactgcatacagcagatatttgaaaccatggtacgtaaatgaagcgattgaaaacaaaagcaataatataatacagacttgcaggtgataattccggatcataagtctaacaagtatcccatactatttacgcaaaaaaaaaatgtatttctagttctagagtccaaaataagattaatagacgaggtttttttatttaaaaaaaaaagagagagagagaatagataactgttttaacttttatctgaagggggagttgctacttttcattcatgtctttaattaattgttaaatatttattagtgatggttgataggaattattgttatttggatttaaatcaccaagcatttacaagtagttaaataagtactattttagaataatcaattagaccaaatacagtactctataatattataaatacatttagattgattcattttattaaataaaacgagagtaccaaacatacaaatgtttaaattataatttttttctctaaaagcttaaaaaattaattcagacttaaacgatagatcagtttaacaagtaaaaagtcattgacataaatatatgacttattcgtccaattgcagaggtaaaatcacgggtctattaaacagtaaatatcgaaacaagtaaaatttgatccgaattgaaggctagtttagttgttggaagcggcagcattagcaatatcaatgacgaaacggtagcgcacatcattcttagcaaggcgctccatagcctcgttgattctgtctggtgtgattaattcaatgtcactcgttatattgtactttgcacaaacctccaacatttcctgcgtctcttttattccccctatgatcccacctttcactgatctcttgcctgcatttcaccaatgttaatcaacatttgttcatcatttttttttaaatcaaccaactaaaaaaggtaattaattaatggatcttcctcaccaagtgtgtttttaaggatattgattaatgaactaaaaagaaaaaacatttattagaaaaatcattggagaatgtaaagaaattatttgtaattccaataagaattttttttcttttgcactagttagctttgccttaattaattagcaaatacaggaagggaaatttaccaaatatcaatgggaaagctggcaattgtaagggcttgtctggtgcacccacgagaaataaggtcccgttgactttgagcaattccaagatgggcaacagagagtgctctgccgaaacagtgtccaatatgaaatctatgcttctccttgcagcctgagtgtcaccagagaaactaaatcatgtttacatagccttcacccatttattatcctaaagcccaaatgcatcaaaccatatatatacttttttaatgctgtgaaatgtagaggcaaacaattgcattacaaacctgtaattgtttagggttggagctgacaatgaaatcatcagcacccaaacgctgcttagcttcagcctccttggagggagaagtgctgatgacggtaacatggtggccgaaagccttgccaaatttaacggcaatgtgcccaaggcctcccagaccaaccaccccaatcttctttcccggtgatgctaccaaatcgtggtctttcaaaggattgaacactgttattcctgcgcatagcagaggagccgctgcatccattgctaggttttccggaatgtgtaccacatatctacacatcattttttcttaatcaaatataaattattctctcaaaaaagaattttttatagacaaagttcaaaacctgactttaaataattattaatttatcaacaaaactacttattttaatactaccaagcattagtgtagtgggggttggttgtcttcaacgaccactgcgatgcctttaaaagcaacgtgagtttcaagataatgtcttcttccaagtatcaatttcatttatggataataactactctaagtctaagataaattggtactaataaaaatatttttaaaaaataaactacaaatatttattgcataaacaagtaaaattattcaatggaagtcaagaatttactttaagcaattcaaaatttattaataaaagtacttataaaaatgagatctaacaattgcaaccaatcataaaaccgcaattgataatttaggc

SEQ ID NO: 17 - DNA, 2565 bp, Glycine max
  modified_base (397)..(500): a, c, t, g, unknown or other
agtacatttactttaaaaggaaaagatttattcgaactctagtatataaagttacaaatatgctacttggttcattgactccgaaattgaagcacccttagtttaaagaacctagattttgtggtgaaaagctaaaaactctaggattcagtgtcacctatttttacagagagagaaaaaaaaaactgtgtataaacagtcttacattataatttgtgattgactaataagattactttatatcattgtaacattaaactctattttatattatgtgtatattagtaggagaggattgcacggagaaaactagaaaggagaaaataagtttaaaaggaaatatgaagaaagaaaagaaaaggacacagttgacatgaaatttctcaaaatttagagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtttctttgcacgattgaattatttcttttaactatccttagcactcatccaaatatgtaaagagaactttctctactttctctcctatttctatcaatccaaaatccaaataacacatcttcctcaccttatttctactctatttctcatcctcctatttctggtaatagcatcctatattagcatctttctttctacttgttcagcattaaagttaaaagcacccgatccaagccatctttgctatgtatcgctcataaagaaaggtggtcaagcaatttgatttgtatctgccttttgcatgcttgtacactcgcattttgcccgtttttcacttagagtctgcatgtgaaattgtgaatgaaaaatgagtgcaaaaaataaacgagtcaacaatagtaacatgatgtgatgcttttgaatttgtaaggcattctcttcttttatgttaccttgaattgtttctgaagatctttaatgtattcaacagccaagtccaacatgtctgctgtgttggtttgctgcaaagagcatcaagaatgttaaatttccaatacaactacatcgcatgtgacatataatttacatattccacaatcaaacgtgcttttatgtgtcttaccttgtccatgtttgggacaagctcttgtaattttctcattctttcactgatccgagttcttctcacctgaaaaatgacttgcatctcagaatcaacttttacaaattacttcatgaattcaacatagtctagataatatcttcttaatttatttattgttttggtataggaagggactgataattactacaatccattcataaaaaacaataaaactacttcctcccaatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatcaattaaattctaaagacaaaattctaccctctcagcaatgcttcgaggatgagtagcacagcctcgctttgctctgattttacaaggaacagaatcagggaactgaagcaacttcttcatagtaatcatctctgctgaagtttttggtaaactcaagtggtgtgataacatattaacttggtttcctagctctccattctgtgtacaacattttatttgtattaagaaaatgaaatgaaagtggaacagaaaaataagaaaagtgagcacttaattaagcattgtatacatacctgaatttcatcagaaaacatcttttcattgctacttcttcctcttttcaagccactgagattttctgagagttgtggagtatcattccaagaaccataggggaatccagggccatattgttgagtatcattaccttcatgccttgtatcatcatcaggactagttgcctcaatgtcttcattccctatttcggagatctgcgacaacattccgagcgaagaagcatttcttggtgagaagctaacctgattcttcaatccgttaatgcatggactaagttcaccattactgccattcaccccaccatagtttccaacacctttcatggtggcaaaccctgccatttacaaattcataactttcagtagccatttataagaatggataaattcatcttaacaacattctacccctcaaaaagaactttgtcttcacaaaataagatccagggtaggatatgtaccctcaaattgaagttactagaaccgtaagtataaagcacaagcttaaatttccaatcttgtgtctctaattacattagtgacaaaagttaaagcataagagagctgagatgaatacagtaaatgtcaagcataggtaaatttcactttggcgagagtttgaatacaaggaatatcatctctctctcaggtatagactatttggaacatcagattctaaataaaacataaattgttcaaaatgtctaccccagaatcattactttctattttcatacacta

SEQ ID NO: 18 - DNA, 1704 bp, Glycine max
ttttcaaatcccataaccacacacacaaatccatccaagatttcccccttgtaagagagagagataaaagaaaatcagcgtcctacagcaccccaccatgggaacccaccaagcacttgtttttatcatgtttttccatcctatatatgacccataagcacacacaagtgtcattcacagccattctttatctctcatttttttaggctcataaatcttttttatgggtttcagggtcactactactctgtctctgctctccctgttttggctcttgtccctgcaccaagcaagagctgcaaagttccacaatgtgagtggcttgagagggaaaaagccagttgtagcaaatgggtgcaatttgttcctgggaagttgggtggttgacacgtcttatcctctctatgactcttcaacatgccccttcattgatcccgagtttgattgccaaaagtatggaagacctgacaaacagtacctcaaatacgcttggaaacctgattcatgtgccatacccaggttcgatggtgcggctttcctgaacagttggaggggtaagaagataatgtttgtgggtgactcactgagtctcaacatgtgggaatcactgtcctgtatgattcacgcgtcggtgccaaatgccaagaccggctttttgagaaaagaatcactgtccactgtgaccttccaggactatggagtaactatacagctataccgcacaccgtatttggtagacataattcgtgaaaatgtggggcgagtgcttacattggactccattgttgctggaaatgcgtggaaaggcatggacatgctgattttcaactcgtggcattggtggacccacactggaaaatctcaggggtgggattatattagagatggacccaatcttgtgaagaacatggaccgtttggaggcctacaataaaggattaaccacttgggctaattgggttgatcagaatgttgatccctccaaaactaaagtcttctttcaaggcatttctcctacacactatcagggcaaggattggaatcaaccaaaaagaagctgtagtggagagcttcaaccattgtctggatcaacatatccagcaggtctacctccagcaactaccatattgaacaatgtgttaaggaagatgagtactccagtttatctacttgatataacactcctctcacaattaaggaaggatgctcacccttctgcttatagtggggaccatgcaggcaatgactgcagccactggtgcctaccaggattaccggacacttggaaccagctcctatatgcagctctcaccaaatgaaggtgaaggccccatacaaattgatccttttcttcatcccttggttatatattcagctagtgtttttagaatttctttattatttgggggatatatagatactacagaggggtgaaactcaattgtaaaattatatataggattcttggaagaataatttacaaaacaaaataagttccttagagatcaaaattgttgcatactaagatgagtactttgtttacaagggttcgtaaactaattttattgaatcacagaatgatgttgaattctatcattggttgatgcacggatcatgtataacatcccatacttgtgacagctacagggttaatcgtaactatgaaactgtttccttccttctgagaaagaaagggtcttattgc

SEQ ID NO: 19 - DNA, 20 bp, Artificial Sequence
  Description of Artificial Sequence Synthetic forward primer for AB052784
atccgacagacgcattctca

SEQ ID NO: 20 - DNA, 20 bp, Artificial Sequence
  Description of Artificial Sequence Synthetic reverse primer for AB052784
agaggaacatgccaaagcct

SEQ ID NO: 21 - DNA, 20 bp, Artificial Sequence
  Description of Artificial Sequence Synthetic forward primer for AF022780
agtccaacccaaccctcaag

SEQ ID NO: 22 - DNA, 20 bp, Artificial Sequence
  Description of Artificial Sequence Synthetic reverse primer for AF022780
gcgttgatgaggatgctgtc

SEQ ID NO: 23 - DNA, 20 bp, Artificial Sequence
  Description of Artificial Sequence Synthetic forward primer for BI944059
accaccccagataatgaggc

SEQ ID NO: 24 - DNA, 20 bp, Artificial Sequence
  Description of Artificial Sequence Synthetic reverse primer for BI944059
ggttgacaccagccattttg

SEQ ID NO: 25 - DNA, 20 bp, Artificial Sequence
  Description of Artificial Sequence Synthetic forward primer for U13987
agtccaacccaaccctcaag

SEQ ID NO: 26 - DNA, 20 bp, Artificial Sequence
  Description of Artificial Sequence Synthetic reverse primer for U13987
gcgttgatgaggatgctgtc

SEQ ID NO: 27 - DNA, 21 bp, Artificial Sequence
  Description of Artificial Sequence Synthetic forward primer for D86929
tgaatcgctgtatagcctccc

SEQ ID NO: 28 - DNA, 20 bp, Artificial Sequence
  Description of Artificial Sequence Synthetic reverse primer for D86929
ggaaacctgttcagtgtggc

SEQ ID NO: 29 - DNA, 20 bp, Artificial Sequence
  Description of Artificial Sequence Synthetic forward primer for BE823110
cagccacttgaggtgaagga

SEQ ID NO: 30 - DNA, 20 bp, Artificial Sequence
  Description of Artificial Sequence Synthetic reverse primer for BE823110
gattgtggaaatgcaagcca

SEQ ID NO: 31 - DNA, 20 bp, Artificial Sequence
  Description of Artificial Sequence Synthetic forward primer for FG999662
gtggaatgctgatgactggg

SEQ ID NO: 32 - DNA, 20 bp, Artificial Sequence
  Description of Artificial Sequence Synthetic reverse primer for FG999662
gcctgcagagtccagttcct

SEQ ID NO: 33 - DNA, 21 bp, Artificial Sequence
  Description of Artificial Sequence Synthetic forward primer for CX710871
gcaggtctacctccagcaact

SEQ ID NO: 34 - DNA, 19 bp, Artificial Sequence
  Description of Artificial Sequence Synthetic reverse primer for CX710871
agtggctgcagtcattgcc

SEQ ID NO: 35 - DNA, 20 bp, Artificial Sequence
  Description of Artificial Sequence Synthetic forward primer for AF055369
agtccaacccaaccctcaag

SEQ ID NO: 36 - DNA, 20 bp, Artificial Sequence
  Description of Artificial Sequence Synthetic reverse primer for AF055369
gcgttgatgaggatgctgtc

SEQ ID NO: 37 - DNA, 20 bp, Artificial Sequence
  Description of Artificial Sequence Synthetic forward primer for CS226295
agcctcagcaccatcctttc

SEQ ID NO: 38 - DNA, 20 bp, Artificial Sequence
  Description of Artificial Sequence Synthetic reverse primer for CS226295
tatgataaccggtggcttgg

SEQ ID NO: 39 - DNA, 20 bp, Artificial Sequence
  Description of Artificial Sequence Synthetic forward primer for BU550410
ggatcaagaccgcgagtaca

SEQ ID NO: 40 - DNA, 21 bp, Artificial Sequence
  Description of Artificial Sequence Synthetic reverse primer for BU550410
cagcctcttgaggtcttctgg

SEQ ID NO: 41 - DNA, 20 bp, Artificial Sequence
  Description of Artificial Sequence Synthetic forward primer for EV281281
agtgccctcaacctgacctc

SEQ ID NO: 42 - DNA, 20 bp, Artificial Sequence
  Description of Artificial Sequence Synthetic reverse primer for EV281281
gacgaatgcatttgggacag

SEQ ID NO: 43 - DNA, 20 bp, Artificial Sequence
  Description of Artificial Sequence Synthetic forward primer for EV276436
agtccattgtcttggcctca

SEQ ID NO: 44 - DNA, 20 bp, Artificial Sequence
  Description of Artificial Sequence Synthetic reverse primer for EV276436
ctagcgcgcacacttaaagc

SEQ ID NO: 45 - DNA, 19 bp, Artificial Sequence
  Description of Artificial Sequence Synthetic forward primer for EF551167
aaggatgatgatgcggtgg

SEQ ID NO: 46 - DNA, 20 bp, Artificial Sequence
  Description of Artificial Sequence Synthetic reverse primer for EF551167
gcatcgtcaaattccacgtc

SEQ ID NO: 47 - DNA, 19 bp, Artificial Sequence
  Description of Artificial Sequence Synthetic forward primer for EH258681
agcaaatcctcgtgaccca

SEQ ID NO: 48 - DNA, 21 bp, Artificial Sequence
  Description of Artificial Sequence Synthetic reverse primer for EH258681
ggtggagccaagttaagatcg

SEQ ID NO: 49 - DNA, 20 bp, Artificial Sequence
  Description of Artificial Sequence Synthetic forward primer for CX709057
aaatcatcagcacccaaacg

SEQ ID NO: 50 - DNA, 20 bp, Artificial Sequence
  Description of Artificial Sequence Synthetic reverse primer for CX709057
acgatttggtagcatcaccg

SEQ ID NO: 51 - DNA, 23 bp, Artificial Sequence
  Description of Artificial Sequence Synthetic forward primer for CX702069
atcatcaggactagttgcctcaa

SEQ ID NO: 52 - DNA, 19 bp, Artificial Sequence
  Description of Artificial Sequence Synthetic reverse primer for CX702069
ggtggggtgaatggcagta